(19) United States
(12) Patent Application Publication
(10) Pub. No.: US 2016/0030369 A1
(43) Pub. Date: Feb. 4, 2016

(54) FKBP52 TARGETING AGENT PHARMACEUTICAL COMPOSITIONS

(71) Applicants: THE UNITED STATES OF AMERICA AS REPRESENTED BY THE SECRETARY, DEPARTMENT OF HEALTH AND HUMAN SERV, Bethesda, MD (US); TEXAS SOUTHERN UNIVERSITY, Houston, TX (US); THE BOARD OF REGENTS OF THE UNIVERSITY OF TEXAS SYSTEM, Austin, TX (US)

(72) Inventors: Huan Xie, Bellaire, TX (US); Su Liang, Houston, TX (US); Leonard M. Neckers, Bethesda, MD (US); Marc B. Cox, El Paso, TX (US)

(21) Appl. No.: 14/775,822

(22) PCT Filed: Mar. 13, 2014
(86) PCT No.: PCT/US2014/025678

§ 371 Date: Sep. 14, 2015

Related U.S. Application Data
(60) Provisional application No. 61/788,716, filed on Mar. 15, 2013.

(51) Int. Cl. A61K 31/167; A61K 31/192; A61K 47/40; A61K 47/10; A61K 47/26; A61K 9/127; A61K 31/4741

(52) U.S. Cl. CPC A61K 31/167; A61K 9/127; A61K 31/192; A61K 31/4741; A61K 9/1271; A61K 47/10; A61K 47/26; A61K 47/40

(57) ABSTRACT

Liposomes comprising an FKBP52 targeting agent (FTA) are disclosed. Pharmaceutical compositions comprising an FTA, a solvent, and a surfactant are disclosed. Pharmaceutical compositions comprising a cyclodextrin and/or a derivative thereof and an FTA are also disclosed. Method of detecting one or more compounds in a sample by liquid chromatography/tandem mass spectrometry (LC/MS/MS), methods of treating or preventing cancer, benign prostatic hyperplasia (BPH), prostatic intraepithelial neoplasia (PIN), prostatitis, enlarged prostate, or insulin-independent diabetes, and methods of inhibiting spermatogenesis or fertilized oocyte implantation in a mammal are also provided.

### BACKGROUND OF THE INVENTION

Normal prostate growth and maintenance may depend on androgens acting through the androgen receptor (AR). AR expression is believed to be involved in a variety of different diseases, including, for example, prostate cancer. The 52 kDa FK506 binding protein (FKBP52) is a specific positive regulator of AR, glucocorticoid receptor (GR), and progesterone receptor (PR) signaling. Some small molecules, including MJC13, can inhibit FKBP52-mediated potentiation of AR signaling and target the AR binding function. MJC13 has high potency and selectivity for AR, and has been shown to effectively block AR-dependent gene expression in cellular models of prostate cancer at micromolar concentrations (De Leon et al., Proc. Natl. Acad. Sci. USA, 108(29): 11878-83 (2011)). Accordingly, MJC13 has potential for as an anti-cancer drug, particularly for the treatment of prostate cancer.

Nevertheless, obstacles to the development of an effective pharmaceutical formulation comprising FKBP52 targeting agents exist. For example, the extremely low aqueous solubility of MJC13 may cause undesirable precipitation of MJC13 upon combination with even small amounts of water.

Accordingly, there is a need for improved formulations comprising FKBP52 targeting agents.

### BRIEF SUMMARY OF THE INVENTION

An embodiment of the invention provides a liposome comprising: (a) a lipid bilayer; (b) at least one liposome stabilizer; and (c) one or more compounds selected from the group consisting of

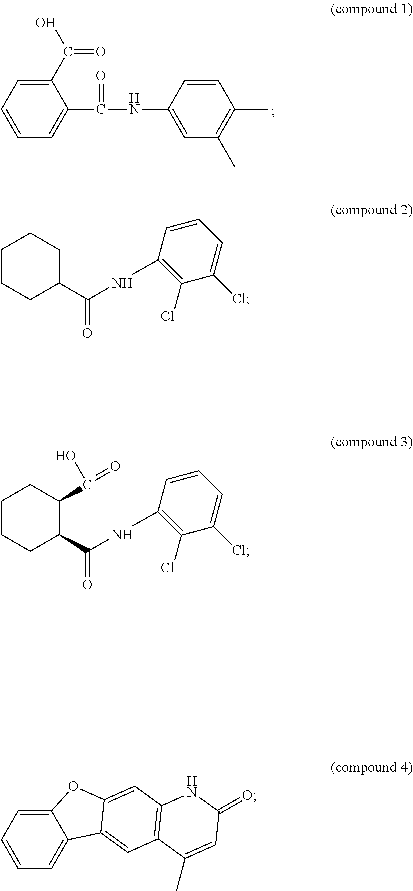 [SMILES: Cc1ccc(NC(=O)c2ccccc2C(=O)O)cc1C ; O=C(Nc1cccc(Cl)c1Cl)C1CCCCC1 ; O=C(Nc1cccc(Cl)c1Cl)[C@H]1CCCC[C@H]1C(=O)O ; Cc1cc(=O)[nH]c2cc3oc4ccccc4c3cc12]

and pharmaceutically acceptable salts, solvates, stereoisomers, derivatives, and hydrates thereof.

An embodiment of the invention provides a pharmaceutical composition comprising: (a) a solvent selected from the group consisting of polyethylene glycol (PEG), alcohol, a polar aprotic solvent, and combinations thereof; (b) a surfactant; and (c) one or more compounds selected from the group consisting of

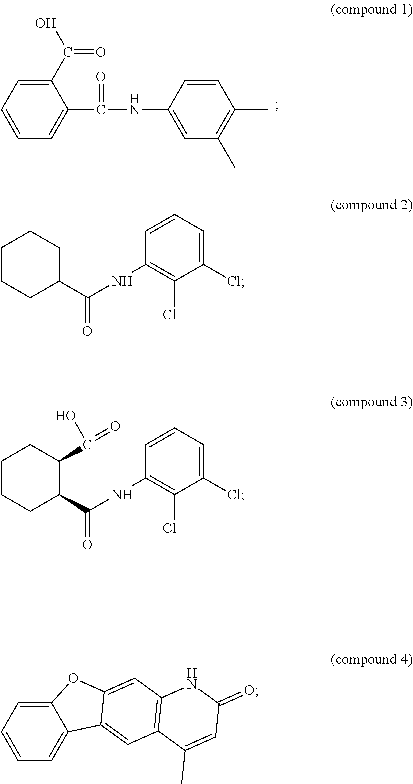 [SMILES: Cc1ccc(NC(=O)c2ccccc2C(=O)O)cc1C ; O=C(Nc1cccc(Cl)c1Cl)C1CCCCC1 ; O=C(Nc1cccc(Cl)c1Cl)[C@H]1CCCC[C@H]1C(=O)O ; Cc1cc(=O)[nH]c2cc3oc4ccccc4c3cc12]

and pharmaceutically acceptable salts, solvates, stereoisomers, derivatives, and hydrates thereof, wherein the pharmaceutical composition comprises a volume/volume (v/v) ratio of (a):(b) of from about 0.5:1 to about 1:5.

Another embodiment of the invention provides a pharmaceutical composition comprising: (a) a solvent selected from the group consisting of polyethylene glycol (PEG), alcohol, a polar aprotic solvent, and combinations thereof; (b) a cyclodextrin and/or a derivative thereof; and (c) one or more compounds selected from the group consisting of

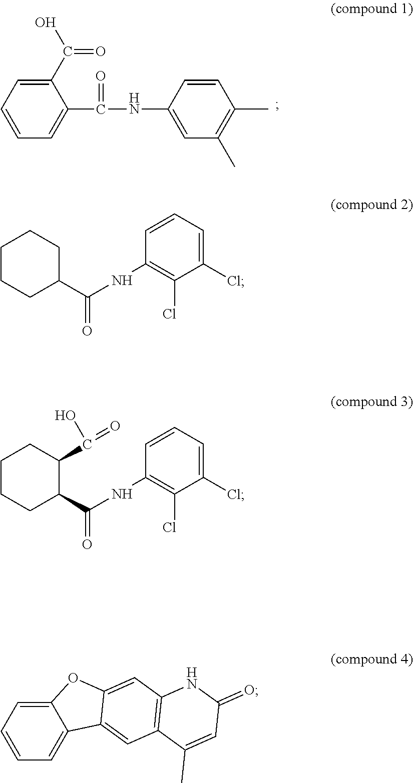 [SMILES: Cc1ccc(NC(=O)c2ccccc2C(=O)O)cc1C ; O=C(Nc1cccc(Cl)c1Cl)C1CCCCC1 ; O=C(Nc1cccc(Cl)c1Cl)[C@H]1CCCC[C@H]1C(=O)O ; Cc1cc(=O)[nH]c2cc3oc4ccccc4c3cc12]

and pharmaceutically acceptable salts, solvates, stereoisomers, derivatives, and hydrates thereof, wherein the pharmaceutical composition comprises a volume/volume (v/v) ratio of (a):(b) of from about 0.5:1 to about 1:5.

Still another embodiment of the invention provides a method of detecting one or more compounds in a sample by liquid chromatography/tandem mass spectrometry (LC/MS/MS), the method comprising: (a) preparing a sample comprising the one or more compounds; (b) separating a first portion of the sample comprising the one or more compounds from a second portion of the sample by liquid chromatography; (c) ionizing the first portion of the sample, separating the ions according to a mass-to-charge ratio, detecting the ions, and generating one or more spectra by tandem mass spectrometry; and (d) determining the presence of the one or more compounds in the sample when the spectra includes a mass peak associated with the compound, wherein the one or more compounds are selected from the group consisting of

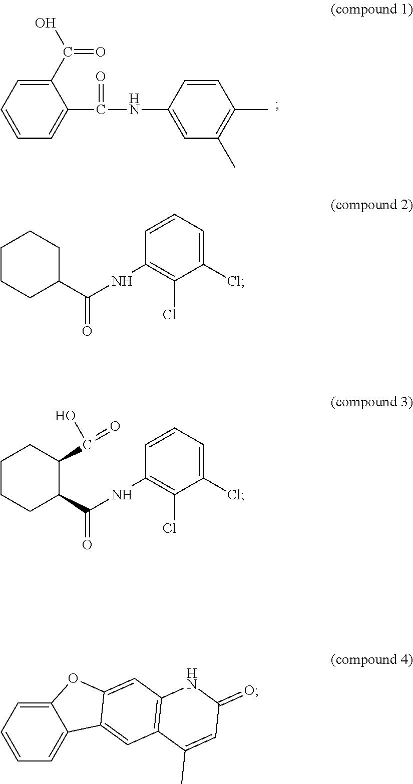 [SMILES: Cc1ccc(NC(=O)c2ccccc2C(=O)O)cc1C ; O=C(Nc1cccc(Cl)c1Cl)C1CCCCC1 ; O=C(Nc1cccc(Cl)c1Cl)[C@H]1CCCC[C@H]1C(=O)O ; Cc1cc(=O)[nH]c2cc3oc4ccccc4c3cc12]

and pharmaceutically acceptable salts, solvates, and stereoisomers thereof

Additional embodiments of the invention provide methods of inhibiting cancer cell growth, treating or preventing a condition, and methods of inhibiting spermatogenesis or fertilized oocyte implantation in a mammal.

### BRIEF DESCRIPTION OF THE SEVERAL VIEWS OF THE DRAWING(S)

FIG. 1 is a graph showing the signal intensity in counts per second (cps) for the mass-to-charge ratio (m/z, Da) of a compound 2 (also referred to as “MJC13”) precursor ion and an MJC13 product ion as measured by product ion scan spectra.

FIGS. 2-4 are representative LC/MS/MS chromatograms showing signal intensity (cps) at retention time (min) for a blank rat plasma spiked with MJC13 (500 ng/mL) with IS (FIG. 2), a rat plasma sample obtained 24 hours after a 2 mg/kg single intravenous (IV) bolus dose of the MJC13 co-solvent formulation PT4 with IS (FIG. 3), and a rat urine sample obtained two hours after a 2 mg/kg single IV bolus dose of the MJC13 co-solvent formulation PT4 with IS (FIG. 4).

FIG. 5 is a graph showing the observed (circles) and predicted (solid line) plasma concentrations (ng/mL) of MJC13 over time (minutes) after a 2 mg/kg single bolus dose of Formulation PT4 in a single rat.

FIG. 6 is a graph showing the observed (circles) and predicted (solid line) plasma concentrations (ng/mL) of MJC13 in a logarithmic scale over time (minutes) after a 2 mg/kg single bolus dose of Formulation PT4 in a single rat.

FIG. 7A is a graph showing the plasma concentration vs. time curve (mean±SD, n=8) of MJC13 after a 2 mg/kg single IV bolus dose of the MJC13 co-solvent formulation PT4 (Y axis is in log₁₀ scale).

FIG. 7B is a graph showing the cumulative urinary excretion vs. time curve (mean±SD, n=4) of MJC13 after a 2 mg/kg single IV bolus dose of the MJC13 co-solvent formulation PT4 (cumulative urinary excretion is expressed as the percentage of the administered dose).

FIG. 8 is a graph showing the tumor volume (mm³) in human prostate cancer xenograft mice treated with the PT4 formulation of MJC13 (squares) or control (circles) over time (days following treatment).

FIG. 9 is a graph showing the plasma drug concentration (ng/mL) of MJC13 at various time points (minutes) after injection of a co-solvent (triangles), non-PEGylated liposome (diamonds), or PEGylated liposome (circles) MJC13 formulation into rats.

### DETAILED DESCRIPTION OF THE INVENTION

It has been discovered that liposomes comprising an FKBP52 targeting agent (FTA) may, advantageously, increase any one or more of water solubility, dissolution rate, stability and bioavailability of the FTA. Accordingly, an embodiment of the invention provides a liposome comprising: (a) a lipid bilayer; (b) at least one liposome stabilizer; and (c) one or more compounds selected from the group consisting of

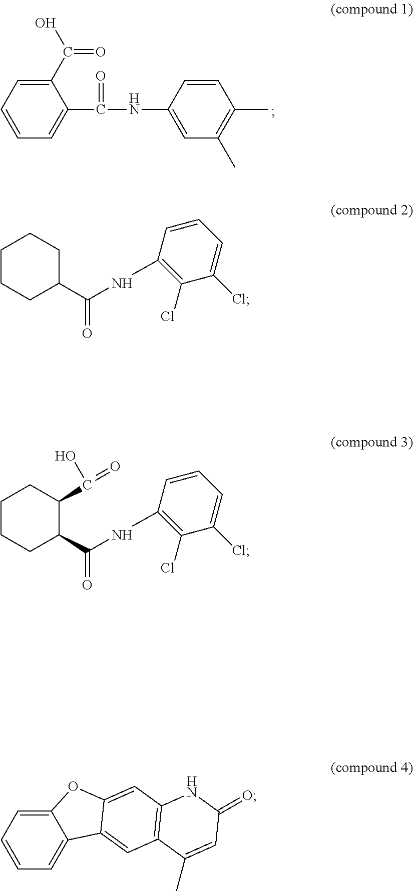 [SMILES: Cc1ccc(NC(=O)c2ccccc2C(=O)O)cc1C ; O=C(Nc1cccc(Cl)c1Cl)C1CCCCC1 ; O=C(Nc1cccc(Cl)c1Cl)[C@H]1CCCC[C@H]1C(=O)O ; Cc1cc(=O)[nH]c2cc3oc4ccccc4c3cc12]

and pharmaceutically acceptable salts, solvates, stereoisomers, derivatives, and hydrates thereof.

Liposomes are vesicles composed of at least one lipid bilayer surrounding a core. The “lipid bilayer,” as used herein, means a membrane composed of two layers of amphiphilic lipid molecules. An amphiphilic lipid has a hydrophilic head and a hydrophobic tail. When amphiphilic lipids are exposed to water, they arrange themselves into a two-layered sheet (a bilayer) with the hydrophobic tails positioned in the hydrophobic interior of the bilayer and the hydrophilic heads positioned at the exterior surfaces of the bilayer. The bilayer has a first hydrophilic exterior surface facing toward the exterior of the liposome and a second hydrophilic exterior surface facing toward the interior of the liposome.

In an embodiment of the invention, the lipid bilayer has first and second hydrophilic surfaces and a hydrophobic interior positioned between the hydrophilic first and second surfaces, and the compound selected from the group consisting of compounds 1-4 and pharmaceutically acceptable salts, solvates, stereoisomers, derivatives, and hydrates thereof is positioned in the hydrophobic interior of the lipid bilayer.

The inventive liposomes provide many advantages. For example, the inventive liposomes may, advantageously, escape premature renal clearance (e.g., clearance by the kidneys before a therapeutic effect can be provided), increase the concentration of the FTA in the plasma, and provide a longer circulation time as compared to, for example, a solvent or co-solvent formulation. The inventive liposomes may protect the FTA from premature metabolism by the host. The inventive liposomes may also, advantageously, target delivery of the FTA to a tumor site and decrease the amount of FTA that is disadvantageously diverted to peripheral tissues. Without being bound to a particular theory or mechanism, passive delivery to a tumor site may be provided by the enhanced permeability and retention (EPR) effect (Maeda et al., J. Control. Release, 65(1-2): 271-84 (2000); Arias et al., Curr. Drug Targets, 12(8): 1151-65 (2011)). Accordingly, the inventive liposomes may make it possible to provide any one or more of reduced dose of FTA administered, increased duration of action of the FTA, and decreased side effects.

In an embodiment of the invention, the lipid bilayer comprises any suitable amphiphilic lipid. The hydrophobic tail (also known as the lipid portion) of the amphiphilic lipid may have, for example, about 14 to about 50 carbon atoms or about 16 to about 24 carbon atoms. In an embodiment, the lipid may be, for example, a phospholipid. Suitable phospholipids may include, for instance, phosphatidyl choline, phosphatidyl glycerol, phosphaphatidyl inositol, phosphatidyl ethanolamine, and combinations thereof. Preferably, the lipid bilayer comprises one or more phospholipids selected from the group consisting of egg phosphatidylcholine (PC), egg phosphatidylglycerol (PG), soy PC, hydrogenated soy PC, didecanoylphosphatidyl choline (DDPC), dilauroylphosphatidyl choline (DLPC), dimyristoylphosphatidyl choline (DMPC), dipalmitoylphosphatidylcholine (DPPC), disaturated phosphatidylcholine (DSPC), dioctanoylphosphatidyl choline (DOPC), palmitoyl oleoyl phosphatidyl choline (POPC), decylphosphatidylcholine (DEPC), dioleoylphosphatidyl ethanolamine (DOPE), dilauroylphosphatidyl ethanolamine (DLPE), dihexanoylphosphatidyl choline (DHPC), dibutyrylphosphatidyl choline (DBPC), and combinations thereof. An especially preferred amphiphilic lipid is egg PC.

The liposome may comprise any suitable liposome stabilizer. The liposome stabilizer may strengthen the bilayer, decrease its permeability, or both. In an embodiment, the liposome comprises at least one liposome stabilizer selected from the group consisting of cholesterol, dimyristoylphosphatidyl glycerol (DMPG), dipalmitoyl phosphatidylglycerol (DPPG), distearoyl phosphatidylglycerol (DSPG), palmitoyl-oleoyl-phosphatidylglycerol (POPG), and combinations thereof. A preferred liposome stabilizer is cholesterol.

The liposome may also comprise one or more compounds selected from the group consisting of compounds 1-4 and pharmaceutically acceptable salts, solvates, stereoisomers, derivatives, and hydrates thereof. The liposome may comprise any suitable amount of FTA. The amount of FTA may be as described herein with respect to other aspects of the invention.

In a preferred embodiment, the liposome comprises compound 2, egg PC, and cholesterol. In an embodiment, the lipid bilayer does not comprise poly(ethylene) glycol (PEG) (also referred to herein as “non-PEGylated liposome”).

The non-PEGylated liposome may comprise the FTA, lipid(s), and liposome stabilizer in any suitable amount. In an embodiment, the liposome may comprise (a) the compound selected from the group consisting of compounds 1-4 and pharmaceutically acceptable salts, solvates, stereoisomers, derivatives, and hydrates thereof, (b) lipid(s), and (c) liposome stabilizer(s) in a molar ratio of about 1:2:1 to 1:>2:>1. In an embodiment, the liposome comprises (a) the compound selected from the group consisting of compounds 1-4 and pharmaceutically acceptable salts, solvates, stereoisomers, derivatives, and hydrates thereof, (b) lipid(s), and (c) liposome stabilizer(s) in a molar ratio of about 1:7.1:3.6.

In an embodiment of the invention, the lipid bilayer comprises phospholipid that comprises at least one PEG (also referred to herein as “a PEGylated phospholipid”). In this regard, the liposome may comprise one or more PEG molecules attached to the first hydrophilic exterior surface at the exterior of the liposome (also referred to herein as a “PEGylated liposome”). The PEG may be covalently attached to the hydrophilic head of the amphiphilic lipid in any suitable manner. For example, the PEG may be covalently attached to the hydrophilic head of the amphiphilic lipid via an amine group.

Providing PEG at the exterior surface of the liposome may provide many advantages. For example, the PEG at the exterior surface of the liposome may reduce uptake of the liposome by macrophages. In this regard, the PEG at the exterior surface of the liposome may, therefore, provide any one or more of prolonged circulation time, increased plasma drug level, and increased duration of action of the FTA.

The PEG may be any suitable PEG. In an embodiment, the phospholipid comprises at least one PEG selected from the group consisting of PEG 350, PEG 550, PEG 750, PEG 1000, PEG 2000, PEG 3000, PEG 5000, methoxy (m) PEG 350, mPEG 550, mPEG 750, mPEG 1000, mPEG 2000, mPEG 3000, mPEG 5000, and combinations thereof. A preferred PEG is mPEG 5000. In an embodiment, the lipid bilayer comprises any one or more of 1,2-distearoyl-sn-glycero-3-phosphoethanolamine-N-[methoxy(polyethylene glycol)-5000] (DSPE-mPEG 5000); N-Carbonyl-methoxypolyethyleneglycol 2000)-1,2-distearoyl-sn-glycero-3-phosphoethanolamine (DSPE-020CN); N-Carbonyl-methoxypolyethyleneglycol 5000)-1,2-distearoyl-sn-glycero-3-phosphoethanolamine (DSPE-050CN); N-Carbonyl-methoxypolyethyleneglycol 2000)-1,2-dipalmitoyl-sn-glycero-3-phosphoethanolamine (PP-020CN); and N-Carbonyl-methoxypolyethyleneglycol 2000)-1,2-dimyristoyl-sn-glycero-3-phosphoethanolamine (PM-020CN). In an especially preferred embodiment, the lipid bilayer comprises DSPE-mPEG 5000.

The PEGylated liposome may comprise the FTA, lipid(s), liposome stabilizer(s), and phospholipid comprising PEG in any suitable amount. In an embodiment, the liposome may comprise (a) the compound selected from the group consisting of compounds 1-4 and pharmaceutically acceptable salts, solvates, stereoisomers, derivatives, and hydrates thereof, (b) phospholipid(s), (c) liposome stabilizer(s), and lipid(s) comprising PEG in a molar ratio of about 1:2:1:0 to 1:>2:>1:>0. In an embodiment, the liposome comprises (a) the compound selected from the group consisting of compounds 1-4 and pharmaceutically acceptable salts, solvates, stereoisomers, derivatives, and hydrates thereof, (b) lipid(s), (c) liposome stabilizer(s), and (d) lipid(s) comprising PEG in a molar ratio of about 1:7.1:3.6:0.6.

The liposomes may be prepared in any suitable manner. In an embodiment, a method of preparing liposomes comprises combining the FTA, amphiphilic lipid(s), liposome stabilizer(s), and optionally PEGylated lipid(s) in a suitable solvent. The solvent may be, for example, any one or more of chlorinated solvents (e.g., chloroform), diethyl ether, methanol, ethanol, ethyl acetate, methanol, and combinations thereof. The method may further comprise evaporating the solvent and preparing vesicles by sonication, high-speed homogenization, or pressure filtration through a membrane having uniform-size pores. The method may further comprise passing the vesicles through an extruder to a desired size. The liposome may be any suitable size. For example, the liposome may have a size of from about 25 nm to about 500 nm, preferably from about 70 nm to about 150 nm. The liposomes may also be, for example, any one or more of multilamellar vesicles (MLV), small unilamellar vesicles (SUV), and large unilamellar vesicles (LUV).

In an embodiment, the liposome may comprise additional pharmaceutical agents in addition to the FTA. Examples of additional pharmaceutical agents that may be included in the liposome include: carcinostatic agents such as adriamycin, daunomycin, mitomycin, cisplatin, vincristine, epirubicin, methotrexate, 5-Fu (5-fluorouracil) and aclacinomycin; toxins such as ricin A and diphtheria toxin; and antisense RNA.

An embodiment of the invention provides a pharmaceutical composition comprising any of the liposomes described herein and a pharmaceutically acceptable carrier such as, for example, those described herein with respect to other aspects of the invention. The pharmaceutically acceptable carrier may be any carrier suitable for combination with liposomes. In an embodiment, the liposomes are not combined with a carrier, and may be administered to a mammal directly. The liposomes may be administered in any suitable manner such as, for example, by injection, inhalation, orally, or topically. Preferably, the liposomes are administered by injection.

It has also been discovered that pharmaceutical compositions comprising an FTA and a volume/volume (v/v) ratio of solvent:surfactant of from about 0.5:1 to about 1:5 may, advantageously, increase any one or more of water solubility, dissolution rate, stability and bioavailability of the FTA. Accordingly, an embodiment of the invention provides a pharmaceutical composition comprising: (a) a solvent selected from the group consisting of polyethylene glycol (PEG), alcohol, a polar aprotic solvent, and combinations thereof; (b) a surfactant; and (c) one or more compounds selected from the group consisting of

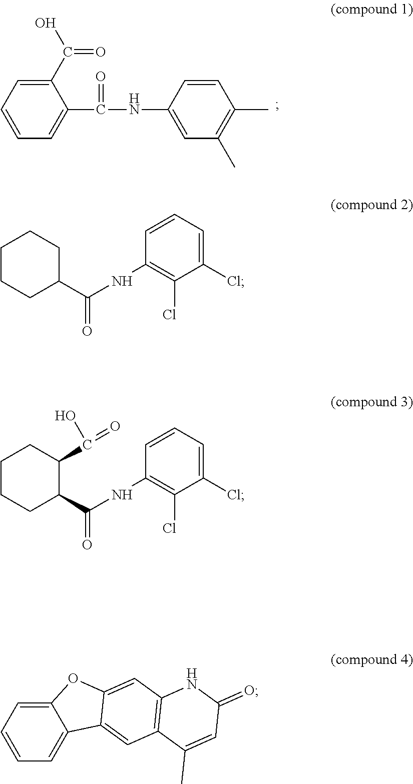 [SMILES: Cc1ccc(NC(=O)c2ccccc2C(=O)O)cc1C ; O=C(Nc1cccc(Cl)c1Cl)C1CCCCC1 ; O=C(Nc1cccc(Cl)c1Cl)[C@H]1CCCC[C@H]1C(=O)O ; Cc1cc(=O)[nH]c2cc3oc4ccccc4c3cc12]

and pharmaceutically acceptable salts, solvates, stereoisomers, derivatives, and hydrates thereof, wherein the pharmaceutical composition comprises a volume/volume (v/v) ratio of (a):(b) of from about 0.5:1 to about 1:5.

An embodiment of the invention provides a pharmaceutical composition comprising a solvent selected from the group consisting of polyethylene glycol (PEG), alcohol, a polar aprotic solvent, and combinations thereof. In an embodiment, the solvent is polyethylene glycol (PEG). The PEG is not limited and may include any PEG suitable for pharmaceutical applications. The PEG may comprise, for example, any one or more of PEG 200, PEG 300, PEG 400, PEG 500, PEG 600, and combinations thereof. In a preferred embodiment, the PEG is PEG 400.

In an embodiment, the solvent is an alcohol. The alcohol may be any alcohol suitable for pharmaceutical compositions. Examples of suitable alcohols may include, but are not limited to, ethanol, isopropanol, and hexadecyl alcohol. Preferably, the alcohol is ethanol.

In an embodiment, the solvent is a polar aprotic solvent. Polar aprotic solvents may accept hydrogen bonds, may dissolve many salts, and lack an acidic hydrogen. Polar aprotic solvents may have high dielectric constants and high dipole moments. The polar aprotic solvent may be any polar aprotic solvent suitable for pharmaceutical compositions. Examples of suitable polar aprotic solvents may include, but are not limited to, acetonitrile, dimethylformamide (DMF), hexamethylphosphoramide (HMPA), tetahydofuran (THF), ethyl acetate (EtOAc), acetone, acetonitrile (MECN), dimethylacetamide (DMA), and dimethyl sulfoxide (DMSO).

An embodiment of the invention provides a pharmaceutical composition comprising a surfactant. The surfactant is not limited and may include any surfactant suitable for pharmaceutical applications. In an embodiment, the surfactant has a hydrophilic-lipophilic balance (HLB) of from about 10 to about 20, preferably from about 14 to about 17. The surfactant may comprise, for example, any one or more of polysorbate 20 (TWEEN 20) (HLB 16.7), polysorbate 40 (TWEEN 40) (HLB 15.6), polysorbate 60 (TWEEN 60) (HLB 14.9), polysorbate 80 (TWEEN 80) (HLB 15.0), BRIJ 97 C₁₈₋₁E₁₀ polyoxyethylene (10) oleyl ether (HLB 12), BRIJ polyethylene glycol octadecyl ether (HLB 12), BRIJ polyethylene glycol hexadecyl ether (HLB 12), BRIJ polyoxyethylene (100) stearyl ether (HLB 18), BRIJ polyoxyethylene (20) oleyl ether (HLB 15), BRIJ polyethylene glycol octadecyl ether (HLB 15), BRIJ polyethylene glycol hexadecyl ether (HLB 16), MYRJ S40 PEG-40 stearate, polyoxyethylene monostearate (MYRJ 49) (HLB 15), mixtures of any of the polysorbates described herein and a SPAN surfactant (e.g., sorbitan monopalmitate (SPAN), sorbitan trioleate (SPAN 85), sorbitan tristearate (SPAN 65)), and combinations thereof. In a preferred embodiment, the surfactant is polysorbate 80.

The pharmaceutical composition may comprise a volume/volume (v/v) ratio of solvent:surfactant of from about 0.5:1 to about 1:5. The v/v ratio of solvent:surfactant of from about 0.5:1 to about 1:5 in the inventive pharmaceutical compositions is believed to advantageously increase any one or more of water solubility, dissolution rate, stability and bioavailability of the FTA. Preferably, the pharmaceutical composition comprises a volume/volume (v/v) ratio of solvent:surfactant of from about 1:1 to about 1:5. For example, the v/v ratio of solvent:surfactant may be about 0.75:1 to about 1:5, about 0.5:1, about 0.5:1.5, about 0.5:2, about 0.5:2.5, about 0.5:3, about 0.5:3.5, about 0.5:4, about 0.5:4.5, about 0.5:5, about 0.75:1, about 0.75:1.5, about 0.75:2, about 0.75:2.5, about 0.75:3, about 0.75:3.5, about 0.75:4, about 0.75:4.5, about 0.75:5, about 1:1, about 1:1.5, about 1:2, about 1:2.5, about 1:3, about 1:3.5, about 1:4, about 1:4.5, or about 1:5. Preferably, the v/v ratio of solvent:surfactant is about 1:1.

The pharmaceutical composition may comprise one or more FKBP52 targeting agents (FTAs). In an embodiment, the FTA is a compound selected from the group consisting of

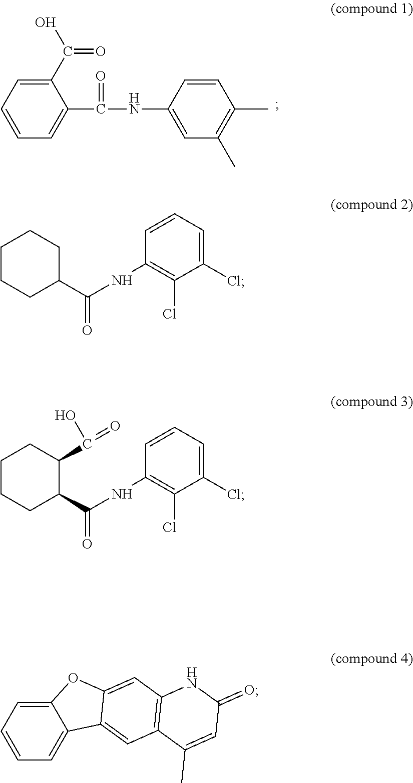 [SMILES: Cc1ccc(NC(=O)c2ccccc2C(=O)O)cc1C ; O=C(Nc1cccc(Cl)c1Cl)C1CCCCC1 ; O=C(Nc1cccc(Cl)c1Cl)[C@H]1CCCC[C@H]1C(=O)O ; Cc1cc(=O)[nH]c2cc3oc4ccccc4c3cc12]

and pharmaceutically acceptable salts, solvates, stereoisomers, derivatives, and hydrates thereof. Preferably, the FTA is

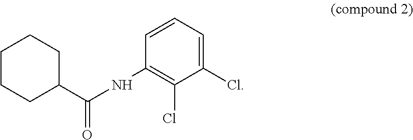 [SMILES: O=C(Nc1cccc(Cl)c1Cl)C1CCCCC1]

The pharmaceutical composition may comprise any suitable amount of FTA. In an embodiment, the pharmaceutical composition may comprise the FTA in a concentration of about 1 mg/mL to about 15 mg/mL, about 2 mg/mL to about 14 mg/mL, about 3 mg/mL to about 13 mg/mL, about 4 mg/mL to about 12 mg/mL, about 5 mg/mL to about 11 mg/mL, about 6 mg/mL to about 10 mg/mL, about 7 mg/mL to about 9 mg/mL, or about 7.5 mg/mL. In a preferred embodiment, the pharmaceutical composition comprises the FTA in a concentration of from about 5 to about 8 mg/mL.

It has also been discovered that pharmaceutical compositions comprising an FKBP52 targeting agent (FTA) and a volume/volume (v/v) ratio of solvent:cyclodextrin and/or a derivative thereof of from about 0.5:1 to about 1:5 may, advantageously, increase any one or more of water solubility, dissolution rate, stability and bioavailability of the FTA. Accordingly, an embodiment of the invention provides a pharmaceutical composition comprising: (a) a solvent selected from the group consisting of polyethylene glycol (PEG), alcohol, a polar aprotic solvent, and combinations thereof; (b) a cyclodextrin and/or a derivative thereof; and (c) one or more compounds selected from the group consisting of

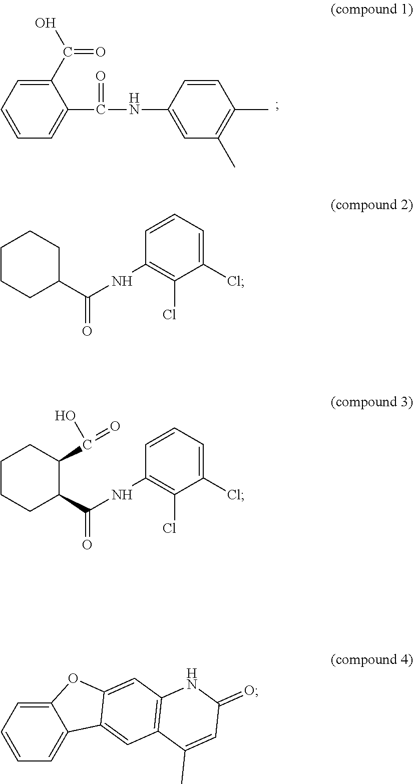 [SMILES: Cc1ccc(NC(=O)c2ccccc2C(=O)O)cc1C ; O=C(Nc1cccc(Cl)c1Cl)C1CCCCC1 ; O=C(Nc1cccc(Cl)c1Cl)[C@H]1CCCC[C@H]1C(=O)O ; Cc1cc(=O)[nH]c2cc3oc4ccccc4c3cc12]

and pharmaceutically acceptable salts, solvates, stereoisomers, derivatives, and hydrates thereof, wherein the pharmaceutical composition comprises a volume/volume (v/v) ratio of solvent:cyclodextrin and/or derivative thereof of from about 0.5:1 to about 1:5.

In an embodiment of the invention, the pharmaceutical composition may comprise a cyclodextrin and/or a derivative thereof. The cyclodextrin and/or a derivative thereof is not limited and may include any cyclodextrin and/or a derivative thereof suitable for pharmaceutical applications. The cyclodextrin and/or a derivative thereof may comprise, for example, any one or more of an α-cyclodextrin, a β-cyclodextrin, a γ-cyclodextrin, and derivatives thereof. In a preferred embodiment, the cyclodextrin and/or a derivative thereof is hydroxypropyl-β-cyclodextrin (HP-β-CD). HP-β-CD is a non-ionic cyclic polysaccharide composed of seven glucose moieties with the shape of a toroid or hollow truncated cone. HP-β-CD is believed to be more water-soluble and more toxicologically benign than natural cyclodextrins. HP-β-CD includes a relatively hydrophobic cavity which may allow water insoluble drugs, such as the FTAs described herein, to form reversible inclusion complexes by non-covalent bonding, mainly hydrogen bonds. The external surface of HP-β-CD is hydrophilic and the substitutions onto the primary and secondary hydroxyl groups with hydroxypropyl groups may, advantageously, increase water solubility.

The pharmaceutical composition may comprise any suitable amount of cyclodextrin and/or a derivative thereof. In an embodiment, the pharmaceutical composition comprises about 10% to about 65% weight/volume (w/v) of cyclodextrin and/or a derivative thereof. For example, the pharmaceutical composition may comprise about 15% to about 60%, about 20% to about 55%, about 25% to about 50%, or about 30% to about 45% cyclodextrin and/or a derivative thereof.

The pharmaceutical composition comprising a cyclodextrin and/or a derivative thereof may further comprise a solvent. The solvent may be as described herein with respect to other aspects of the invention.

The pharmaceutical composition comprising cyclodextrin and/or a derivative thereof may comprise a volume/volume (v/v) ratio of solvent:cyclodextrin and/or a derivative thereof of from about 0.5:1 to about 1:5. The v/v ratio of solvent:cyclodextrin and/or a derivative thereof of from about 0.5:1 to about 1:5 in the inventive pharmaceutical compositions is believed to advantageously increase any one or more of water solubility, dissolution rate, stability and bioavailability of the FTA. Preferably, the pharmaceutical composition comprises a volume/volume (v/v) ratio of solvent:cyclodextrin and/or a derivative thereof of from about 1:1 to about 1:5, about 0.5:1.5, about 0.5:2, about 0.5:3, or about 0.5:4. For example, the v/v ratio of solvent:cyclodextrin and/or a derivative thereof may be about 0.75:1 to about 1:5, about 0.5:1, about 0.5:1.5, about 0.5:2, about 0.5:2.5, about 0.5:3, about 0.5:3.5, about 0.5:4, about 0.5:4.5, about 0.5:5, about 0.75:1, about 0.75:1.5, about 0.75:2, about 0.75:2.5, about 0.75:3, about 0.75:3.5, about 0.75:4, about 0.75:4.5, about 0.75:5, about 1:1, about 1:1.5, about 1:2, about 1:2.5, about 1:3, about 1:3.5, about 1:4, about 1:4.5, or about 1:5. Preferably, the v/v ratio of solvent:cyclodextrin and/or a derivative thereof is about 1:1.

The pharmaceutical composition comprising cyclodextrin and/or a derivative thereof may comprise an FTA. The FTA may be as described herein with respect to other aspects of the invention. The pharmaceutical composition may comprise any suitable amount of FTA. In an embodiment, the pharmaceutical composition may comprise the FTA in a concentration of about 0.5 to about 10 mg/mL. In an embodiment, the pharmaceutical composition may comprise the FTA in a concentration of about 1 mg/mL to about 9 mg/mL, about 2 mg/mL to about 8 mg/mL, about 3 mg/mL to about 7 mg/mL, or about 4 mg/mL to about 6 mg/mL.

The inclusion of a cyclodextrin and/or a derivative thereof in the pharmaceutical composition may, advantageously, make it possible to increase any one or more of water solubility, dissolution rate, stability and bioavailability without including a surfactant in the pharmaceutical composition. Accordingly, in an embodiment of the invention, the pharmaceutical composition comprising a cyclodextrin and/or a derivative thereof and an FTA does not comprise a surfactant, including any of the surfactants described herein with respect to other aspects of the invention. In an embodiment of the invention, the pharmaceutical composition comprising a cyclodextrin and/or a derivative thereof and an FTA does not comprise polysorbate 80.

As used herein, the term “derivative” includes, but is not limited to, ether derivatives, acid derivatives, amide derivatives, ester derivatives and the like. Methods of preparing these derivatives are known to a person skilled in the art. For example, ether derivatives are prepared by the coupling of the corresponding alcohols. Amide and ester derivatives are prepared from the corresponding carboxylic acid by a reaction with amines and alcohols, respectively.

In an embodiment of the invention, the pharmaceutical composition may comprise a hydrate of the FKBP52 targeting agent (FTA). The term “hydrate” includes but is not limited to hemihydrate, monohydrate, dihydrate, trihydrate and the like. Hydrates of the FTA compounds may be prepared by contacting the FTA with water under suitable conditions to produce the hydrate of choice.

In an embodiment of the invention, the pharmaceutical composition may comprise a solvent addition form (“solvate”) of the FKBP52 targeting agents (FTAs). Some compounds have a tendency to trap a fixed molar ratio of solvent molecules in the crystalline solid state, thus forming a solvate. When the FTA is placed in a system in which a certain solvent is brought to a vapor form, in some situations, the compound, together with the molecules of the solvent, forms a crystal. The material formed by crystallization of the FTA and the solvent in a three-dimensional order is called a solvate herein. The solvent can be associated with a crystalline solid form of an FTA in various ways. The interaction can be due to weak binding (e.g., hydrogen bonding, van der Waals, and dipole-dipole) or by entrapment (e.g., liquid inclusion).

A solvate can be formed by a variety of methods, many of which are known in the art. An FTA can be combined with one or more solvents by any suitable method (e.g., crystallization, lyophilization, film coating, spray drying, suspension, wetting, grinding, vapor sorption, etc.). For example, an FTA can be combined with a particular solvent(s) and heated to boiling. The solution can then be slowly cooled to allow formation of the solvate crystals. Cooling can occur at room temperature or at a reduced temperature (e.g., an ice bath and/or refrigerated conditions). Controlling the temperature can be influential in the formation of solvates. Typically a lower temperature favors solvate formation. The formed solvate can be characterized by analytical methods such as thermogravimetric analysis (TGA), differential scanning calorimetry (DSC) alone or with infrared (IR) and/or mass spectrometry, x-ray powder diffraction, moisture sorption experiments, hot-stage polarized light microscopy, or a combination of these methods. Various techniques to prepare solvates are known in the art. See, e.g., J. Keith Guillory, “Generation of Polymorphs, Hydrates, and Solvates, and Amorphous Solids,” Drugs and the Pharmaceutical Sciences, 95 (Polymorphism in Pharmaceutical Solids): 183-226 (1999); and Greisser, U., “The Importance of Solvates” in Polymorphism, Hilfiker, R., Ed., (Wiley-VCH Verlag GmbH & Co. KGaA: Weinheim, Germany, 2006), pages 211-233.

A solvate means a solvent addition form that contains either stoichiometric or non-stoichiometric amounts of solvent. A stoichiometric solvate implies a fixed, although not necessarily integral, ratio of solvent to compound (e.g., a solvent coordination number of 1, 2, 3, 4, 5, 6, etc.). A preferred solvent coordination number of a stoichiometric solvate is 1. A non-stoichiometric solvate can be an interstitial solid solution or an interstitial co-crystal. The solvent content of a solvate can be any suitable value, including a multiple of the molar compound ratio such that the solvent coordination number is a non-integral number (e.g., 0.1, 0.2, 0.3, 0.4, 0.5, 0.6, 0.7, 0.8, 0.9, 1.1, 1.2, 1.3, 1.4, 1.5, 1.6, 1.7, 1.8, 1.9, etc.). The amount of solvent in the structure generally depends on the partial pressure of the solvent in the environment of the solid and the temperature (See, e.g., Greisser, U., supra).

In an embodiment of the invention, the pharmaceutical composition may comprise a pharmaceutically acceptable salt of the FTA. As used herein, the phrase “salt” or “pharmaceutically acceptable salt” is intended to include nontoxic salts synthesized from the parent compound which contain a basic or acidic moiety by conventional chemical methods. Generally, such salts can be prepared by reacting the free acid or base forms of these compounds with a stoichiometric amount of the appropriate base or acid in water or in an organic solvent, or in a mixture of the two. Generally, nonaqueous media such as ether, ethyl acetate, ethanol, isopropanol, or acetonitrile are preferred. Lists of suitable salts are found in Remington's Pharmaceutical Sciences, 18th ed., Mack Publishing Company, Easton, Pa., 1990, p. 1445, and Journal of Pharmaceutical Science, 66, 2-19 (1977). For example, they can be a salt of an alkali metal (e.g., sodium or potassium), alkaline earth metal (e.g., calcium), or ammonium of salt.

The FTA may be modified by the covalent attachment of water-soluble polymers such as polyethylene glycol, copolymers of polyethylene glycol and polypropylene glycol, carboxymethyl cellulose, dextran, polyvinyl alcohol, polyvinylpyrrolidone or polyproline. The modified FTA may exhibit substantially longer half-lives in blood following intravenous injection as compared to the unmodified FTA. Such modifications may also increase the FTA's solubility in aqueous solution, eliminate aggregation, enhance the physical and chemical stability of the compound, and greatly reduce the immunogenicity and reactivity of the compound. As a result, the desired in vivo biological activity may be achieved by the administration of such polymer-compound abducts less frequently or in lower doses than with the unmodified compound.

The pharmaceutical composition may further comprise additional excipients suitable for water-soluble pharmaceutical compositions. Parenteral vehicles (for subcutaneous, intravenous, intraarterial, or intramuscular injection) may include sodium chloride solution, Ringer's dextrose, dextrose and sodium chloride, and lactated Ringer's. Formulations suitable for parenteral administration may include aqueous and non-aqueous, isotonic sterile injection solutions, which can contain anti-oxidants, bacteriostats, and solutes that render the formulation isotonic with the blood of the intended recipient, and aqueous and non-aqueous sterile suspensions that can include suspending agents, thickening agents, stabilizers, and preservatives.

Intravenous vehicles may include fluid and nutrient replenishers, electrolyte replenishers such as those based on Ringer's dextrose, and the like. Examples are sterile liquids such as water, with or without the addition of other pharmaceutically acceptable adjuvants. In general, water, saline, aqueous dextrose and related sugar solutions are preferred liquid carriers, particularly for injectable solutions.

The pharmaceutical formulations can be presented in unit-dose or multi-dose sealed containers, such as ampoules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets.

The pharmaceutical compositions may be formulated so as to be suitable for any route of administration. For example, the pharmaceutical compositions of the invention may be suitable for any of parenteral, subcutaneous, intravenous, intramuscular, intraarterial, intrathecal, and interperitoneal administration. More than one route can be used to administer the pharmaceutical compositions, and in certain instances, a particular route can provide a more immediate and more effective response than another route.

In an embodiment, the pharmaceutical composition is injectable. The requirements for effective pharmaceutical carriers for injectable compositions are well-known to those of ordinary skill in the art (see, e.g., Pharmaceutics and Pharmacy Practice, J.B. Lippincott Company, Philadelphia, Pa., Banker and Chalmers, eds., pages 238-250 (1982), and ASHP Handbook on Injectable Drugs, Toissel, 4th ed., pages 622-630 (1986)).

An embodiment of the invention provides a method of treating or preventing a condition in a mammal, the method comprising administering to the mammal any of the liposomes or pharmaceutical compositions described herein in an amount effective to treat or prevent the condition in the mammal. The condition may be any condition. In an embodiment, the condition may be cancer. The cancer can be any cancer, including any of acute lymphocytic cancer, acute myeloid leukemia, alveolar rhabdomyosarcoma, bladder cancer, bone cancer, brain cancer, breast cancer (for example, late stage, estrogen-independent breast cancer), cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, chronic lymphocytic leukemia, chronic myeloid cancer, colon cancer, esophageal cancer, cervical cancer, fibrosarcoma, gastrointestinal carcinoid tumor, Hodgkin lymphoma, hypopharynx cancer, kidney cancer, larynx cancer, leukemia, liquid tumors, liver cancer, lung cancer, lymphoma, malignant mesothelioma, mastocytoma, melanoma, multiple myeloma, nasopharynx cancer, non-Hodgkin lymphoma, ovarian cancer, pancreatic cancer, peritoneum, omentum, and mesentery cancer, pharynx cancer, prostate cancer, rectal cancer, renal cancer, skin cancer, small intestine cancer, soft tissue cancer, solid tumors, stomach cancer, testicular cancer, thyroid cancer, ureter cancer, and urinary bladder cancer.

In another embodiment, the condition is an inflammatory condition selected from the group consisting of thyroid disease; sepsis; cardiovascular disease; asthma; lung fibrosis; bronchitis; respiratory infections; respiratory distress syndrome; obstructive pulmonary disease; allergic diseases; multiple sclerosis; infections of the brain or nervous system; dermatitis; psoriasis; skin infections; gastroenteritis; colitis; Crohn's disease; cystic fibrosis; celiac disease; inflammatory bowel disease; intestinal infections; conjunctivitis; uveitis; infections of the eye; kidney infections; autoimmune kidney disease; diabetic nephropathy; cachexia; coronary restenosis; sinusitis, cystitis; urethritis; serositis; uremic pericarditis; cholecystis; vaginitis; hepatitis; pelvic inflammatory disease; vitiligo; alopecia; Addison's disease; Hashimoto's disease; Graves disease; atrophic gastritis/pernicious anemia; acquired hypogonadism/infertility; hypoparathyroidism; multiple sclerosis; Myasthenia gravis; Coombs positive hemolytic anemia; systemic lupus erthymatosis; and Siogren's syndrome.

In an embodiment, the condition is an androgen receptor polyglutamine tract disease. For example, the androgen receptor polyglutamine tract disease may be Kennedy's disease.

It is contemplated that the liposomes and pharmaceutical compositions of the present invention may be useful for the treatment or prevention of any of a variety of hormone-related conditions wherein androgenic, glucocorticoid and/or progesterone activity are upregulated compared to normal levels and downregulation of androgenic, glucocorticoid and/or progesterone activity are believed to provide a therapeutic effect. In this regard, an embodiment of the invention provides a method of treating or preventing a condition in a mammal, the method comprising administering to the mammal any of the liposomes or pharmaceutical compositions described herein in an amount effective to treat or prevent the condition in the mammal, wherein the condition is any of the cancers described herein (e.g., prostate cancer, breast cancer), or benign prostatic hyperplasia (BPH), prostatic intraepithelial neoplasia (PIN) (e.g., high grade PIN), prostatitis, enlarged prostate, metabolic syndrome, or insulin-independent diabetes.

Another embodiment of the invention provides a method of inhibiting cancer cell growth in a mammal, the method comprising administering to the mammal any of the liposomes or pharmaceutical compositions described herein in an amount effective to inhibit cancer cell growth in the mammal. In an embodiment, the cancer cells may be prostate cancer cells or breast cancer cells.

Any of the inventive methods may further comprise administering additional therapeutic agents. In this regard, in an embodiment of the invention, the methods of treating or preventing cancer or inhibiting cancer cell growth may comprise administering any of the liposomes or pharmaceutical compositions described herein in combination with one or more additional chemotherapeutic and/or anti-androgenic agents. The additional chemotherapeutic and/or anti-androgenic agents may include, for example, any of CASODEX (bicalutamide), NILANDRON (nilutamide), flutamide, finasteride, and ketoconazole.

In yet another embodiment, the methods of treating or preventing cancer, prostatic intraepithelial neoplasia (PIN) (e.g., high grade PIN), prostatitis, enlarged prostate, or BPH may comprise administering any of the liposomes or pharmaceutical compositions described herein in combination with one or more additional therapeutic agents such as, for example, 5-alpha-reductase inhibitors (e.g., finasteride or ketoconazole). In one embodiment, the 5-alpha-reductase inhibitor is MK-906, a product of Merck, Sharp & Dohme (McConnell et al., J. Urol., 141:239A (1989)). In another embodiment, the 5-alpha-reductase inhibitor is 17-β-N,N-diethylcarbamoyl-4-methyl-4-aza-5-α-androstan-3-one (4-MA) (Brooks et al., Endocrinology, 109:830-836, (1981); Liang et al., Endocrinology, 112:1460-1468 (1983)). In another embodiment, the 5-alpha-reductase inhibitor is a 4-azasteroid, which can be formed as described in Liang et al., J. Biol. Chem., 259:734-739, (1984); and in Brooks et al., Steroids, 47:1-19, (1986)). In another embodiment, the 5-alpha-reductase inhibitor is a 6-methylene-4-pregnene-3,20-dione, for example, as described (Petrow et al., J. Endocrinol., 95:311-313 (1982)). In yet another embodiment, the 5-alpha-reductase inhibitor is a 4-methyl-3-oxo-4-aza-5-α-pregnane-30(s) carboxylate (Kadohama et al., J. Natl. Cancer Inst., 74:475-486 (1985)).

In an embodiment, the methods of treating or preventing cancer, (e.g., prostate cancer), BPH, prostatic intraepithelial neoplasia (PIN) (e.g., high grade PIN), prostatitis, enlarged prostate, or the methods of inhibiting cancer cell growth may comprise administering any of the liposomes or pharmaceutical compositions described herein in combination with one or more testosterone decreasing compounds. Testosterone decreasing compounds may include, for example, LH-RH agonists. LH-RH agonists include, for example, LUPRON (leuprolide), VIADUR (leuprolide acetate implant), and ZOLADEX (goserelin).

In still another embodiment of the invention, the methods of treating or preventing non-insulin dependent diabetes or metabolic syndrome may comprise administering any of the liposomes or pharmaceutical compositions described herein in combination with an additional therapeutic agent useful in the treatment of non-insulin dependent diabetes or metabolic syndrome such as, for example, one or more from the class of compounds including sulfonylureas, metglitinides, biguanides, thiazolidinediones and DPP-4 inhibitors. Examples of such compounds include metformin, glibenclamide, gliclazide, acarbose, rosiglitazone and pioglitazone.

It is also contemplated that the liposomes and pharmaceutical compositions of the present invention may be useful for diminishing fertility in a mammal. For example, it is believed that an FTA may bind to the FKBP52 protein modulating the androgen receptor and suppress spermatogenesis. In this regard, another embodiment of the invention provides a method of inhibiting spermatogenesis in a male mammal, the method comprising administering to the mammal any of the liposomes or pharmaceutical compositions described herein in an amount effective to inhibit spermatogenesis in the mammal. Another embodiment of the invention provides a method of inhibiting fertilized oocyte implantation in a female mammal, the method comprising administering to the mammal any of the liposomes or pharmaceutical compositions described herein in an amount effective to inhibit fertilized oocyte implantation in the mammal. Embryo implantation in the uterus is a step in mammalian reproduction, requiring preparation of the uterus in order to be receptive to blastocyst implantation. Uterine receptivity, also known as the window of implantation, lasts for a limited period of time, and it is during this period that blastocysts normally implant. The ovarian steroid hormones estrogen and progesterone (P₄) are the primary regulators of this process. The immunophilin FKBP52 serves as a cochaperone for steroid hormone nuclear receptors to govern appropriate hormone action in target tissues. See, Tranguch, S., et al., Proc. Nat. Acad. Sci. USA, 102(40):14326-14331 (2005). It was found that females missing the FKBP52 gene have complete implantation failure due to lack of attainment of uterine receptivity. The overlapping uterine expression of FKBP52 with nuclear progesterone receptor (PR) in wild-type mice together with reduced P₄ binding to PR, attenuated PR transcriptional activity and down-regulation of several P₄-regulated genes in uteri of FKBP52^{-/-} mice, establishes this cochaperone as a potential regulator of uterine P₄ function. Accordingly, it is believed that the inventive liposomes and pharmaceutical compositions may effectively inhibit pregnancy.

For purposes of the invention, the amount or dose of the FTA administered should be sufficient to effect, e.g., a therapeutic or prophylactic response, in the subject over a reasonable time frame. The dose will be determined by the efficacy of the particular FTA and the condition of a human, as well as the body weight of a human to be treated.

The dose of the FTA also will be determined by the existence, nature and extent of any adverse side effects that might accompany the administration of a particular FTA. Typically, an attending physician will decide the dosage of the FTA with which to treat each individual patient, taking into consideration a variety of factors, such as age, body weight, general health, diet, sex, FTA to be administered, route of administration, and the severity of the condition being treated. By way of example, and not intending to limit the invention, the dose of the FTA can be about 0.001 to about 1000 mg/kg body weight of the subject being treated/day, from about 0.01 to about 10 mg/kg body weight/day, about 0.01 mg to about 1 mg/kg body weight/day.

The terms “treat,” and “prevent” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect. In this respect, the inventive methods can provide any amount or any level of treatment or prevention of cancer, BPH, PIN (e.g., high grade PIN), prostatitis, enlarged prostate, metabolic syndrome, or insulin-independent diabetes in a mammal. Furthermore, the treatment or prevention provided by the inventive method can include treatment or prevention of one or more conditions or symptoms of the condition, e.g., cancer, BPH, BPH, PIN (e.g., high grade PIN), prostatitis, enlarged prostate, metabolic syndrome, or insulin-independent diabetes, being treated or prevented. Also, for purposes herein, “prevention” can encompass delaying the onset of the condition, or a symptom or condition thereof.

As used herein, the term “mammal” refers to any mammal including, but not limited to, mammals of the order Logomorpha, such as rabbits; the order Carnivora, including Felines (cats) and Canines (dogs); the order Artiodactyla, including Bovines (cows) and Swines (pigs); or of the order Perssodactyla, including Equines (horses). It is preferred that the mammals are non-human primates, e.g., of the order Primates, Ceboids, or Simoids (monkeys) or of the order Anthropoids (humans and apes). In some embodiments, the mammal may be a mammal of the order Rodentia, such as mice and hamsters. Preferably, the mammal is a non-human primate or a human. An especially preferred mammal is the human.

Another embodiment of the invention provides a method of detecting one or more compounds in a sample by liquid chromatography/tandem mass spectrometry (LC/MS/MS), the method comprising: (a) preparing a sample comprising the one or more compounds; (b) separating a first portion of the sample comprising the one or more compounds from a second portion of the sample by liquid chromatography; (c) ionizing the first portion of the sample, separating ions according to a mass-to-charge ratio, detecting the ions, and generating one or more spectra by tandem mass spectrometry; and (d) determining the presence of the one or more compounds in the sample when the spectra includes a mass peak associated with the compound, wherein the one or more compounds are selected from the group consisting of

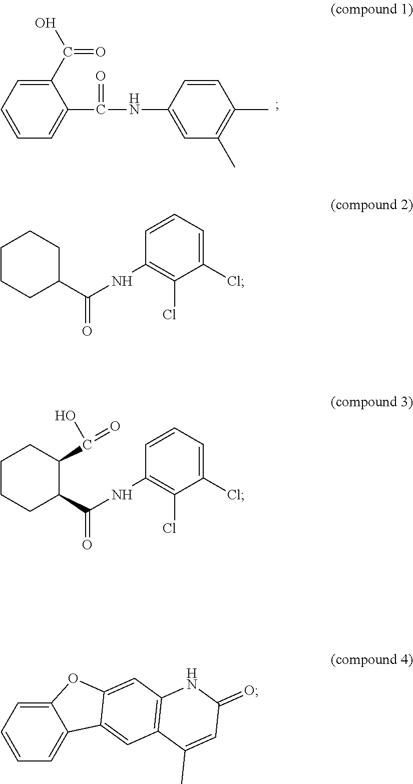 [SMILES: Cc1ccc(NC(=O)c2ccccc2C(=O)O)cc1C ; O=C(Nc1cccc(Cl)c1Cl)C1CCCCC1 ; O=C(Nc1cccc(Cl)c1Cl)[C@H]1CCCC[C@H]1C(=O)O ; Cc1cc(=O)[nH]c2cc3oc4ccccc4c3cc12]

and pharmaceutically acceptable salts, solvates, and stereoisomers thereof. The inventive method advantageously provides a simple, specific, sensitive, and reliable method for the detection and/or quantification of FTA in a sample. Accordingly, it is contemplated that the inventive method may be useful for pharmacokinetic and/or pharmacodynamic studies of FTAs. The method may, advantageously, detect even a trace amount of the FTA in the sample. In an embodiment, the method may comprise detecting the one or more compounds present in the sample at a concentration of about 1 picogram to about 1 nanogram.

The method may comprise preparing a sample comprising the one or more compounds. The sample may be prepared in any manner suitable for analysis by any type of liquid chromatography and/or mass spectrometry (MS), particularly liquid chromatography/tandem mass spectrometry (LC/MS/MS). The sample may be derived from any suitable source. In an embodiment, the sample comprises blood (e.g., blood plasma), or an aqueous solution.

The method may comprise separating a first portion of the sample comprising the one or more compounds from a second portion of the sample by liquid chromatography. The liquid chromatography may be any liquid chromatography known in the art (e.g., high performance liquid chromatography (HPLC) and ÄKTA™ fast protein liquid chromatography (FPLC)). The stationary and mobile phases used for liquid chromatography may be any suitable stationary and mobile phase known in the art and which may be suitable for separation of the particular sample under study.

The method may comprise ionizing the first portion of the sample, separating the ions according to a mass-to-charge ratio, detecting the ions, and generating one or more spectra by tandem mass spectrometry. Ionizing the first portion of the sample, separating the ions according to a mass-to-charge ratio, detecting the ions, and generating one or more spectra by tandem mass spectrometry may be carried out in any suitable manner. For example, the method may comprise loading the separated portion of the sample comprising the one or more compounds into an MS instrument and vaporizing the sample. The method may comprise ionizing the vaporized sample by any one of a variety of methods (e.g., by impacting the sample with an electron beam), which may result in the formation of charged particles (ions). The method may comprise separating the ions according to their mass-to-charge ratio in an analyzer by electromagnetic fields. The method may further comprise detecting the ions and processing the ion signal into mass spectra. The method may comprise determining the presence of the one or more compounds in the sample when the spectra includes a mass peak associated with the compound.

The compound detected or quantified may be a FTA. The FTA may be any of the FTAs described herein with respect to other aspects of the invention.

In some instances, it may be useful to quantify the amount of the one or more compounds in the sample. In this regard, the method may further comprise quantifying the one or more compounds in the sample in any suitable manner.

The following examples further illustrate the invention but, of course, should not be construed as in any way limiting its scope.

### Example 1

This example demonstrates a liquid chromatography-tandem mass spectrometry (LC/MS/MS) method for detection and quantification of compound 2 (also referred to as “MJC13”) in solution, rat plasma, and rat urine.

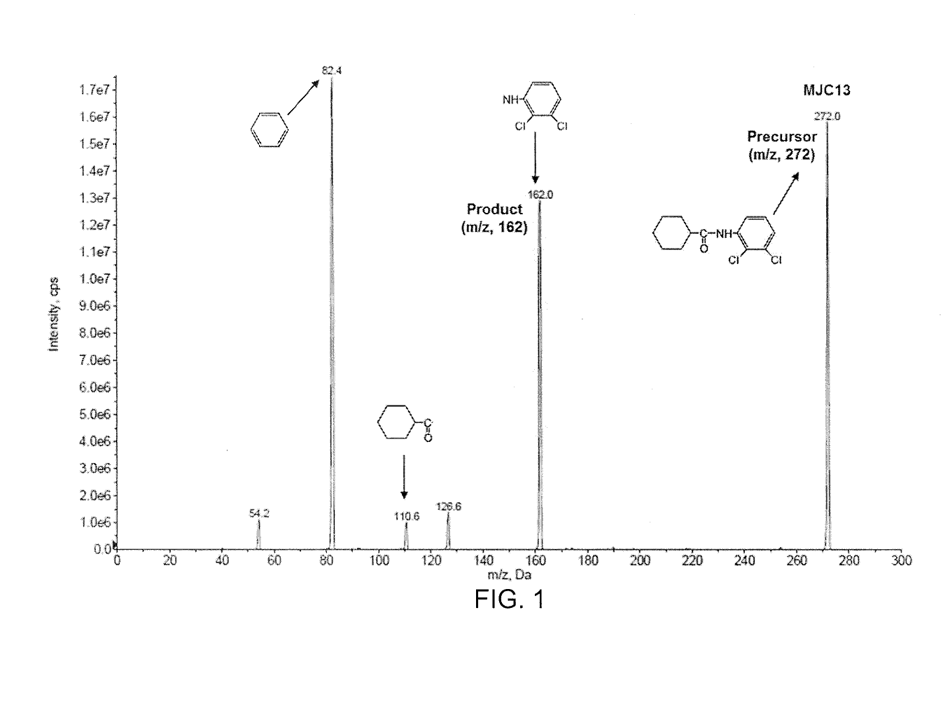

The LC/MS/MS system was controlled and data was acquired by ANALYST software version 1.5. Chromatographic analysis was performed using a 3200 QTRAP LC/MS/MS system (AB SCIEX, Foster City, Calif.), which is a hybrid triple quadrupole linear ion trap equipped with a TURBOIONSPRAY ion source. Pure nitrogen was generated by a Parker Balston Source 5000 TRIGAS Generator (Parker, Cleveland, Ohio). The ion source parameters for mass spectrum were set as follows: ionspray voltage, 4500 V; ion source temperature, 300° C.; nebulizer gas, 60 psi; heater gas, 60 psi; curtain gas, 10 psi; and the collision gas, medium. Itraconazole was used as internal standard (IS) to analyze MJC13 in solution, rat plasma, and rat urine. The product ion scan spectra of MJC13 (compound 2) is presented in FIG. 1. Multiple reaction monitoring (MRM) method in the positive ion mode was used to detect the transition ions from precursor ion to a specific product ion for MJC13 (m/z 272→m/z 162) and itraconazole (m/z 705→m/z 392). The collision energy was set at 23 and 49 V for MJC13 and IS, respectively.

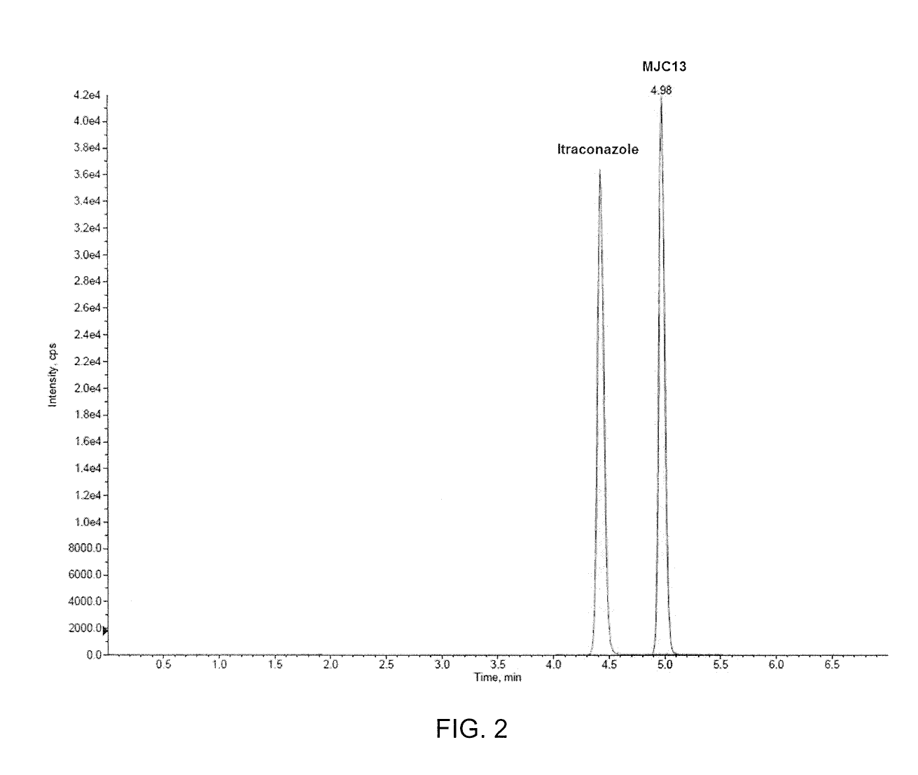

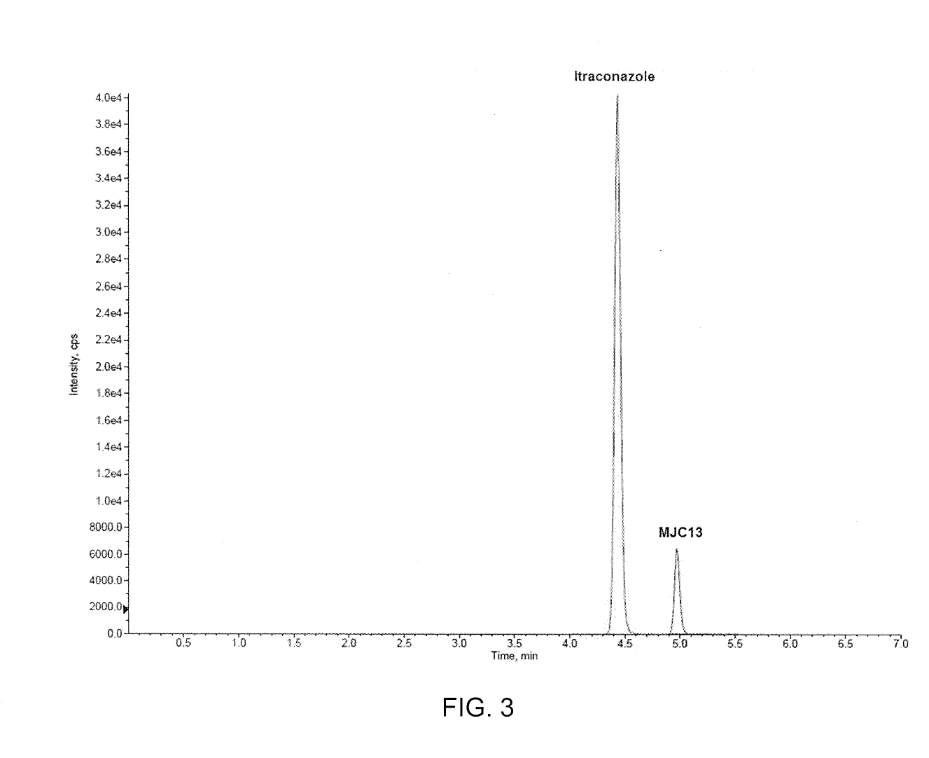

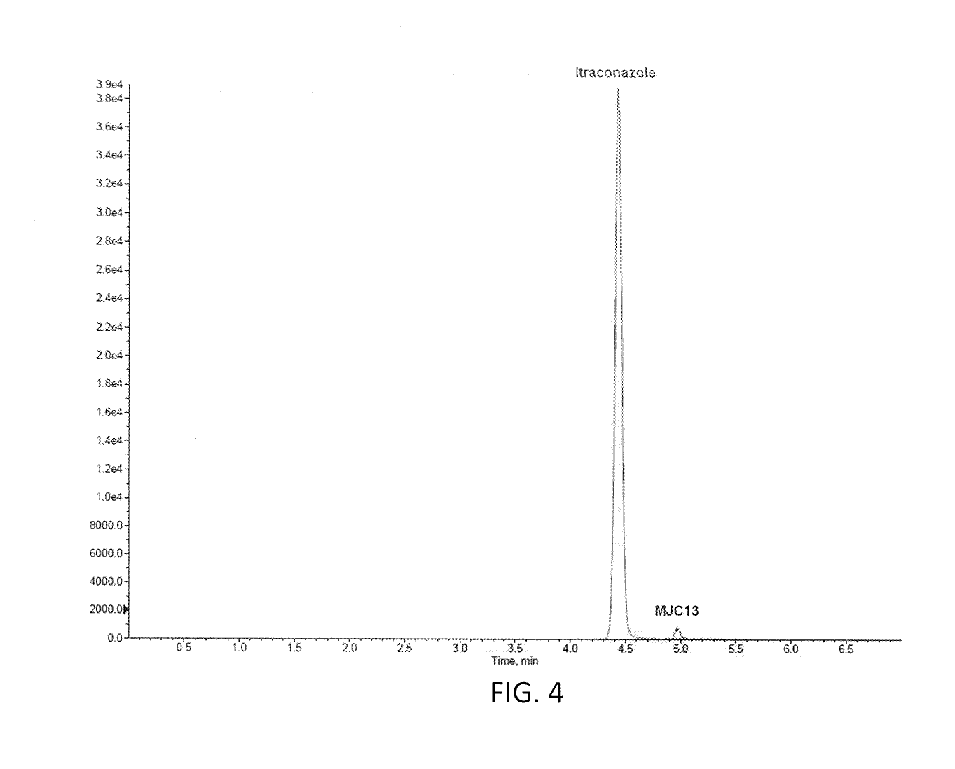

Chromatographic separation was achieved with a Waters XTERRA MS C18 column (3.5 μm, 4.6×150 mm, Milford, Mass.) using an Agilent 1200 series high performance liquid chromatography (HPLC) system (Foster City, Calif.). Mobile phase solvent A was water and solvent B was 0.2% formic acid in acetonitrile. All samples were analyzed under gradient elution: initial, 5% B, 0-1 min, 5-95% B, 1-2 min, 95% B, 2-5 min, 95-5% B, 5-6 min, 5% B, 6-7 min, at a flow rate of 1 mL/minutes (min.). The injection volume was 10 μL. The retention times for MJC13 and IS were 4.98 min and 4.42 min, respectively (FIGS. 2-4).

Stock solution of MJC13 was prepared by dissolving MJC13 in acetonitrile at the concentration of 1 mg/mL. Stock solution of IS was prepared by dissolving itraconazole in acetonitrile at the concentration of 1 mg/mL. The stock solutions were stored at −80° C. prior to use. Standard rat plasma samples of MJC13 were prepared by diluting the stock solution with acetonitrile and spiking in blank rat plasma at seven different concentrations: 1, 10, 50, 100, 200, 500 and 1000 ng/mL. Quality control (QC) rat plasma samples of MJC13 were prepared at low (5 ng/mL), medium (400 ng/mL) and high (800 ng/mL) concentrations in rat plasma. Standard rat urine samples of MJC13 were prepared by diluting the stock solution with acetonitrile and spiking in blank rat urine at six different concentrations: 1, 10, 25, 50, 100 and 200 ng/mL. Quality control (QC) rat urine samples of MJC13 were prepared at low (5 ng/mL), medium (90 ng/mL) and high (180 ng/mL) concentrations in rat urine.

The protein precipitation method was used for the preparation of rat plasma samples. Briefly, an aliquot of rat plasma sample (50 μL) was extracted with 200 pt acetonitrile (containing 200 ng/mL of IS) by vortex for 1 minute. After centrifugation at 13,000 revolutions per minute (rpm) for 10 min, a 200 μL aliquot of the supernatant was transferred to an autosampler vial, and then injected to the LC/MS/MS for quantitative analysis.

An aliquot of rat urine sample (500 μL) was evaporated to dryness under a stream of nitrogen. The residue was reconstituted and extracted with 200 μL acetonitrile (containing 200 ng/mL of IS) by vortex for one min. After centrifugation at 13,000 rpm for 10 min, a 100 μL aliquot of the supernatant was transferred to an autosampler vial, and then injected to the LCMS/MS for quantitative analysis.

Linear calibration curves in solution, rat plasma and rat urine were generated by plotting the peak area ratios of MJC13 to IS vs. seven known MJC13 standard concentrations. Slope, intercept and correlation coefficient of linear regression equation were estimated using least square regression analysis. The lower limit of quantification (LLOQ) was determined based on the signal-to-noise ratio of at least 5:1. The calibration curves for MJC13 in solution, rat plasma, and rat urine were linear in the concentration range of 1-1000, 1-1000, and 1-200 ng/mL, respectively, with correlation coefficient values >0.998.

The intra-day accuracy and precision were determined by analyzing six replicates of QC samples with three different concentrations using a calibration curve constructed on the same day. The inter-day accuracy and precision were determined by analyzing six replicates of QC samples with three different concentrations using calibration curves constructed on three different days. The assay accuracy was reflected by relative error from the theoretical drug concentrations and the assay precision was reflected by the coefficient of variation. The accuracy and precision obtained are summarized in Table 1. These data showed that the accuracy and precision were well within the 15% acceptance range.

TABLE 1
Intra- and inter-day accuracy and precision of
MJC13 LC/MS/MS analysis.

| | | Intra-day (n = 6) | | Inter-day (n = 6) | |
|---|---|---|---|---|---|
| | Concentration | Accuracy | Precision | Accuracy | Precision |
| | (ng/mL) | (RE*, %) | (CV*, %) | (RE, %) | (CV, %) |
| Solution | 5 | 3.25 | 3.76 | 3.25 | 4.23 |
| | 400 | 2.12 | 2.85 | 2.14 | 3.19 |
| | 800 | 1.99 | 2.27 | 1.83 | 2.80 |
| Plasma | 5 | 4.07 | 3.76 | 4.39 | 4.07 |
| | 400 | 2.38 | 2.83 | 2.63 | 3.54 |
| | 800 | 2.65 | 3.47 | 3.92 | 4.79 |
| Urine | 5 | 4.20 | 3.88 | 3.33 | 4.76 |
| | 90 | 3.61 | 3.12 | 4.27 | 3.48 |
| | 180 | 3.95 | 3.54 | 3.83 | 4.35 |

*RE = relative error; CV = coefficient of variation.

This LC/MS/MS method was validated for accuracy and precision in rat plasma over a MJC13 concentration range of 1-1000 ng/mL. In order to analyze the sample with an even high concentration, the accuracy and precision of a sample dilution was conducted. The accuracy and precision of diluted rat plasma samples were determined by analyzing six replicates of 5 times dilution of a plasma sample with 2500 ng/mL of MJC13 and six replicates of 10 times dilution of a plasma sample with 5000 ng/mL of MJC13. The accuracy and precision were 8.93% (relative error) and 8.55% (coefficient of variation), respectively, for 5 times dilution; and 9.24% (relative error) and 8.94% (coefficient of variation), respectively, for 10 times dilution. These data suggested that the concentration estimation from a 5 or 10 times diluted plasma sample was reliable.

The extraction recovery and matrix effect was evaluated by analyzing QC samples at low, medium, and high concentrations. The extraction recovery of MJC13 was calculated according to Equation (Eq.) 1:

$$\% Recovery = \frac{{Response}_{extracted sample}}{{Response}_{post \text{-} extracted spiked sample}} \times 100 \% \qquad Eq . 1$$

where Response_{extracted sample} is the average area count for MJC13, which has been through the extraction process. Response_{post-extracted spiked sample} is the average area count for MJC13 spiked into extracted matrix after the extraction procedure. The average extraction recovery rate obtained by measuring triplicates of QC samples at low, medium and high concentration levels of MJC13 in rat plasma were 94.3±3.9%, 95.7±4.8% and 95.9±3.1%, respectively; and in rat urine were 96.6±3.5%, 96.7±3.7% and 95.4±5.0%, respectively. The high and stable recovery rate is satisfactory.

The matrix effect of MJC13 was calculated according to Eq. 2:

$$\% Matrix effect = ( \frac{{Response}_{post \text{-} extracted spiked sample}}{{Response}_{non \text{-} extracted neat sample}} - 1 ) \times 100 \% \qquad Eq . 2$$

where Response_{post-extracted spiked sample} is the average area count for the MJC13 spiked into extracted matrix after the extraction procedure, and Response_{non-extracted neat sample} is the average area count for the same concentration of MJC13 in neat solution (acetonitrile). A positive result of the percentage matrix effect indicates enhancement of the sample signal, and a negative result indicates suppression of the sample signal. The average percentage matrix effect acquired by measuring triplicates of QC samples at low, medium and high concentration levels of MJC13 in rat plasma were −7.3±4.2%, −9.4±5.8% and −6.3±3.1%, respectively; and in rat urine were −9.5±5.4%, −9.7±6.1% and −8.8±6.5%, respectively. These results suggest that there was no measurable matrix effect that interfered with MJC13 determination in rat plasma and rat urine via this LC/MS/MS method.

Short-term stability (bench-top stability) of MJC13 in rat plasma and rat urine were evaluated by analyzing triplicates of QC samples at low, medium, and high concentrations. Plasma samples were removed from frozen storage (−20° C.) and remained on the bench-top for 2, 4, or 6 hours. All of those samples were compared with freshly prepared samples at the same concentrations. The rat plasma samples displayed 98.6±1.7%, 97.6±2.0% and 97.4±1.2% average MJC13 amount remaining, respectively; and rat urine samples displayed 97.9±2.5%, 99.1±1.9% and 96.7±2.8% average MJC13 amount remaining, respectively. These data indicated that MJC13 in rat plasma and rat urine were stable for at least 6 hours on the bench-top. Another set of plasma samples were stored at −80° C. for 7 days. This set of plasma samples displayed 96.4±1.4% average recoveries, and rat urine samples that were stored at −80° C. for 7 days displayed 95.9±2.7% average MJC13 amount remaining, which suggested that the MJC13 in rat plasma and rat urine were stable in the freezer for at least 7 days.

Freeze-thaw stability of MJC13 in rat plasma and rat urine were evaluated by analyzing QC samples at low, medium, and high concentrations. Plasma samples that were exposed for three cycles of freezing (at −20° C.) and thawing (at room temperature) were compared with freshly prepared samples at the same concentrations. The rat plasma and rat urine samples displayed 96.7±2.0% and 97.5±2.4% average MJC13 amount remaining after the test, respectively. These data indicated that MJC13 in rat plasma and rat urine were stable after three freeze-thaw cycles.

Processed sample stability (on-instrument or autosampler stability) of MJC13 rat plasma extracts and rat urine extracts were evaluated by analyzing QC samples at low, medium, and high concentrations. Two sets of plasma samples were extracted with acetonitrile with and without IS, respectively. The plasma extracts were kept in the autosampler for 2, 4, or 6 hours, before injecting into the LC-MS/MS. All of those samples were compared with freshly prepared samples at the same concentrations. The rat plasma samples that were extracted by acetonitrile with IS displayed 97.6±1.3%, 98.3±1.5% and 96.7±0.7% average recoveries, respectively; and the rat urine samples displayed 95.8±3.2%, 98.6±2.5% and 97.9±1.7% average recoveries, respectively. The samples that were extracted by acetonitrile without IS displayed 96.1±1.3%, 97.6±1.1% and 97.7±2.4% recoveries, respectively; and rat urine samples displayed 97.4±2.2%, 97.1±1.9% and 96.4±3.3% recoveries, respectively. These data indicated that the processed MJC13 rat plasma extracts were stable for at least 6 hours in the autosampler, and were not affected by the presence of the IS.

The stability of MJC13 in rat urine was conducted in the same fashion as in rat plasma.

### Example 2

This example demonstrates the preparation of an intravenous pharmaceutical composition comprising MJC13, polysorbate 80 (TWEEN 80), and polyethylene glycol (PEG) 400.

To investigate the effect of storage temperature on stability, a dry powder of MJC13 was stored at three different temperatures (−20° C., 4° C. and room temperature) and analyzed via LC/MS/MS at various time intervals for up to 1 month to determine the amount of MJC13 remaining. Experiments were conducted in triplicate. The results are summarized in Table 2. After 1 month, the samples stored in three different conditions displayed 97.3±2.5%, 99.7±3.5% and 95.3±2.8% average recoveries, respectively. These data indicate that MJC13 powder was stable at −20° C., 4° C. and room temperature for 1 month.

TABLE 2
MJC13 solid-state stability.

| | Storage | 3-day | 1-week | 2-week | 1-month |
|---|---|---|---|---|---|
| | condition | recovery (%) | recovery (%) | recovery (%) | recovery (%) |
| Solid-state | −20° C. | 99.3 ± 3.5 | 97.7 ± 1.5 | 97.0 ± 1.0 | 97.3 ± 2.5 |
| | 4° C. | 99.3 ± 1.5 | 99.3 ± 0.6 | 100.7 ± 1.5 | 99.7 ± 3.5 |
| | room temperature | 100.3 ± 3.2 | 100.0 ± 3.6 | 101.3 ± 3.8 | 95.3 ± 2.8 |

The solubility of MJC13 was evalulated. Because there is no ionizable functional group on the molecular structure, the solubility of MJC13 was expected to be low in water and barely affected by pH. In this work, the solubility of MJC13 in water, ethanol, propylene glycol, polyethylene glycol 400 (PEG 400), polysorbate 80 (Tween 80), olive oil, peanut oil, soybean oil, CAPTEX 200 and LABRAFAC Lipophile WL1349 was determined by a shake-flask method. Briefly, an excess amount of MJC13 was added to each capped glass bottle containing the selected vehicle and mixed for 48 hours at room temperature using a reciprocating shaker. The samples were centrifuged at 3000 rpm for 10 minutes and were subsequently filtered through a 0.22 μm ultrafiltration unit. The filtrates were analyzed by LC/MS/MS to determine the concentration of drug dissolved. The experiments were conducted in triplicate. The results are summarized in Table 3. As shown in Table 3, the water solubility of MJC13 was only 0.28 μg/mL.

TABLE 3
Solubility of MJC13 in various solvents.

| Solvent | Solubility (mg/mL) |
|---|---|
| Water | (2.8 ± 0.2) × 10⁻⁴ |
| DMSO | 100~200 |
| Ethanol | 35.6 ± 1.8 |
| Propylene glycol | 5.7 ± 0.2 |
| PEG 400 | 23.0 ± 0.7 |
| Polysorbate 80 | 28.2 ± 0.7 |
| Olive oil | 16.2 ± 0.3 |
| Peanut oil | 15.6 ± 0.4 |
| Soybean oil | 17.3 ± 0.5 |
| Captex ® 200 | 31.9 ± 1.6 |
| Labrafac ™ Lipophile WL1349 | 24.0 ± 1.1 |

The lipophilicity of MJC13 was determined as the logarithm of partition coefficient (log P) of the solute between water and 1-octanol. Equal volumes of two-phase solution (water and 1-octanol) was mixed well in a capped glass bottle and then placed in a shaker for 24 hours to make sure that saturation equilibrium had been achieved. MJC13 (1 mL) dissolved in the two-phase solution was added the bottle. Then, MJC13 was partitioned between aqueous and 1-octanol for 72 hours at room temperature using the shake-flask method. The mixture from the bottle was centrifuged at 3000 rpm for 10 minutes and subsequently filtered through a 0.22 μm ultrafiltration unit, and then moved to a separatory funnel for phase separation. The aqueous and 1-octanol phases were analyzed by LC-MS/MS to determine the concentration of drug dissolved. Experiments were conducted in triplicate. The log P was calculated according to Eq. 3:

$$Log P = log \frac{{C}_{0}}{{C}_{w}} \qquad Eq . 3$$

where Cₒ and C_{w} represent the concentrations of MJC13 in 1-octanol and aqueous phase, respectively. The experimental log P of MJC13 between water and 1-octanol was 6.49±0.37.

In vitro rat plasma samples of MJC13 were prepared by diluting the stock solution with acetonitrile and spiking in rat plasma at five different concentrations: 100, 500, 1000, 2000 and 5000 ng/mL to approximate the plasma concentrations of MJC13 in rats after 2 mg/kg intravenous bolus dosing. The plasma was incubated at 37° C. for 1 hours before being transferred to Amicon Ultra-0.5 mL Centrifugal Filters (EMD Millipore Corporation, Billerica, Mass.) for ultrafiltration at 3000 rpm for 10 minutes. Filtrate and nonfiltrate concentrations were determined by LC-MS/MS. The fraction unbound, fᵤ, was determined as Eq. 4:

$${f}_{u} = \frac{{C}_{u}}{{C}_{t}} \qquad Eq . 4$$

where Cᵤ is the unbound concentration and Cₜ is the total concentration. The fᵤ of MJC13 in rat plasma at concentrations of 100, 500, 1000, 2000 and 5000 ng/mL were found to be 98.1±1.5%, 97.6±2.7%, 99.6±1.7%, 97.2±2.8% and 97.9±2.4%, respectively. These data indicate that MJC13 is highly protein bound, and the binding is concentration independent.

The solubility of a non-water-soluble molecule can be increased by the addition of water miscible solvents. Solvents used in combination to increase the solubility of a solute are known as co-solvents. Water miscible co-solvents may be used to formulate intravenous non-water-soluble drugs. However, drug precipitation often occurs upon the addition of the co-solvent mixture to intravenous fluids or blood. In such cases, extremely slow infusion is required to prevent precipitation. Co-solvent systems with various compositions and ratios of dimethyl sulfoxide (DMSO), ethanol, PEG 400 and Tween 80 were prepared with a MJC13 concentration range of from 5-10 mg/mL. Each system was diluted with 0.9% sodium chloride solution (normal saline) at the ratios of 1:1, 1:3, 1:7 and 1:15 (v/v) to evaluate whether MJC13 would precipitate within 4 hours. After 4 hours, no precipitation occurred in the diluted formulation, indicating that the formulation had a good capacity to dissolve MJC13 in an aqueous environment. It is believed that an intravenous dose of this formulation will not cause precipitation at the site of administration. A system with high co-solvent solubility and great aqueous tolerance was selected for further studies. The formulation was selected based on consideration of four factors: (1) MJC13 solubility, (2) MJC13 precipitation upon dilution, (3) solvent toxicity, and (4) formulation stability. The results are summarized in Table 4. The results indicated that: (1) MJC13 has good solubility in DMSO (greater than 100 mg/mL), but the inclusion of DMSO in co-solvent systems cannot prevent MJC13 precipitation upon dilution and will cause undesirable toxic effects; (2) MJC13 has relatively good solubility in ethanol and PEG 400, but the inclusion of ethanol and PEG 400 in co-solvent systems cannot prevent MJC13 precipitation upon dilution without the present of surfactant; (3) the inclusion of Tween 80 in co-solvent systems can prevent MJC13 precipitation upon dilution; (4) either ethanol or PEG 400 combined with a high percentage of Tween 80 can achieve relatively good solubility of MJC13 and prevent MJC13 precipitation upon dilution; (5) considering that ethanol is a highly volatile solvent which may cause the formulation to become unstable during storage, co-solvent systems including PEG 400 and Tween 80 are more preferred; (6) the final MJC13 co-solvent formulation included PEG 400 and Tween 80 (1:1, v/v) with an MJC13 concentration of 7.5 mg/mL; and (7) the co-solvent formulation can be diluted with normal saline to the desirable MJC13 concentration before IV bolus or IV infusion administration, which is well accepted in clinical settings.

TABLE 4
MJC13 co-solvent systems: composition, ratio and precipitation upon
dilution with normal saline at the ratio of 1:1, 1:3, 1:7 and 1:15
(v/v) after 4 hours. Note Y indicates Yes and N indicates No.

| | Composition and ratio of solvent (v/v) | | | | | Precipitation upon dilution with | | | |
|---|---|---|---|---|---|---|---|---|---|
| | | | | Tween | MJC13 Conc. | normal saline (v/v) | | | |
| Label | DMSO | Ethanol | PEG 400 | 80 | (mg/mL) | 1:1 | 1:3 | 1:7 | 1:15 |
| P1 | — | — | 1 | — | 5 | Y | Y | Y | Y |
| E1 | — | 1 | — | — | 5 | Y | Y | Y | Y |
| EP1 | — | 1 | 9 | — | 5 | Y | Y | Y | Y |
| EP2 | — | 3 | 7 | — | 5 | Y | Y | Y | Y |
| EP3 | — | 1 | 1 | — | 5 | Y | Y | Y | Y |
| EP4 | — | 7 | 3 | — | 5 | Y | Y | Y | Y |
| EP5 | — | 9 | 1 | — | 5 | Y | Y | Y | Y |
| DP1 | 1 | — | 9 | — | 5 | Y | Y | Y | Y |
| DEP1 | 1 | 1 | 8 | — | 5 | Y | Y | Y | Y |
| DEP2 | 1 | 3 | 6 | — | 5 | Y | Y | Y | Y |
| DEP3 | 1 | 5 | 4 | — | 5 | Y | Y | Y | Y |
| DEP4 | 1 | 7 | 2 | — | 5 | Y | Y | Y | Y |
| DE1 | 1 | 9 | — | — | 5 | Y | Y | Y | Y |
| DP2 | 3 | — | 7 | — | 5 | Y | Y | Y | Y |
| DEP5 | 3 | 1 | 6 | — | 5 | Y | Y | Y | Y |
| DEP6 | 3 | 3 | 4 | — | 5 | Y | Y | Y | Y |
| DEP7 | 3 | 5 | 2 | — | 5 | Y | Y | Y | Y |
| DE2 | 3 | 7 | — | — | 5 | Y | Y | Y | Y |
| DP3 | 5 | — | 5 | — | 5 | Y | Y | Y | Y |
| DEP8 | 5 | 1 | 4 | — | 5 | Y | Y | Y | Y |
| DEP9 | 5 | 3 | 2 | — | 5 | Y | Y | Y | Y |
| DE3 | 5 | 5 | — | — | 5 | Y | Y | Y | Y |
| PT1 | — | — | 9 | 1 | 5 | Y | Y | Y | Y |
| PT2 | — | — | 7 | 3 | 5 | N | N | Y | Y |
| PT3 | — | — | 5 | 5 | 5 | N | N | N | N |
| PT4* | — | — | 5 | 5 | 7.5 | N | N | N | N |
| PT5 | — | — | 5 | 5 | 10 | N | Y | Y | Y |
| ET1 | — | 9 | — | 1 | 5 | Y | Y | Y | Y |
| ET2 | — | 7 | — | 3 | 5 | N | N | Y | Y |
| ET3 | — | 5 | — | 5 | 5 | N | N | N | N |
| ET4 | — | 5 | — | 5 | 7.5 | N | N | N | Y |
| DPT1 | 1 | — | 8 | 1 | 5 | Y | Y | Y | Y |
| DPT2 | 1 | — | 6 | 3 | 5 | N | N | N | Y |
| DPT3 | 1 | — | 4 | 5 | 5 | N | N | N | N |
| DPT4 | 1 | — | 4 | 5 | 7.5 | N | N | N | N |
| DPT5 | 1 | — | 4 | 5 | 10 | N | N | Y | Y |
| DPT6 | 3 | — | 6 | 1 | 10 | Y | Y | Y | Y |
| DPT7 | 3 | — | 4 | 3 | 10 | N | N | Y | Y |
| DPT8 | 3 | — | 2 | 5 | 10 | N | N | N | Y |

*Formulation PT4 including PEG 400 and Tween 80 (1:1, v/v) with a MJC13 concentration of 7.5 mg/mL provided a suitable co-solvent formulation.

To investigate the effect of storage temperature on the formulation stability, Formulation PT4 was stored at three different temperatures (−20° C., 4° C. and room temperature) and analyzed via LC/MS/MS at various time intervals for up to 1 month to determine the amount of MJC13 present. Experiments were conducted in triplicate. The results are summarized in Table 5. After 1 month, stored in three different conditions, the samples displayed 98.0±6.1%, 97.3±1.5% and 96.7±1.5% average recoveries, respectively. These data indicate that Formulation PT4 was stable at −20° C., 4° C. and room temperature for at least 1 month. Co-solvent formulation PT4 was chosen for further study.

TABLE 5
Formulation PT4 stability

| | Storage | 3-day | 1-week | 2-week | 1-month |
|---|---|---|---|---|---|
| | condition | recovery (%) | recovery (%) | recovery (%) | recovery (%) |
| Formulation | −20° C. | 101.0 ± 2.0 | 99.0 ± 1.0 | 99.0 ± 2.6 | 98.0 ± 6.1 |
| PT4 | 4° C. | 97.7 ± 1.2 | 96.3 ± 2.1 | 100.3 ± 2.5 | 97.3 ± 1.5 |
| | 20° C. | 98.0 ± 2.0 | 98.3 ± 2.1 | 98.3 ± 3.5 | 96.7 ± 1.5 |

### Example 3

This example demonstrates the pharmacokinetics of Formulation PT4 administered to rats.

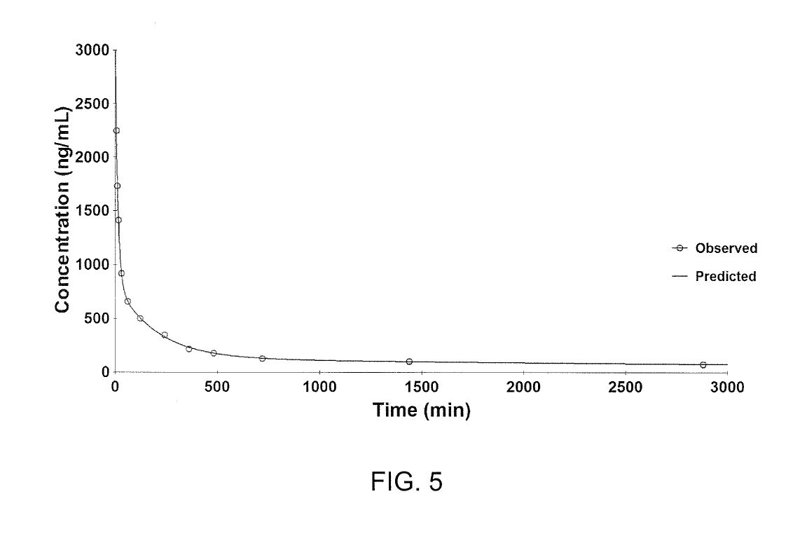

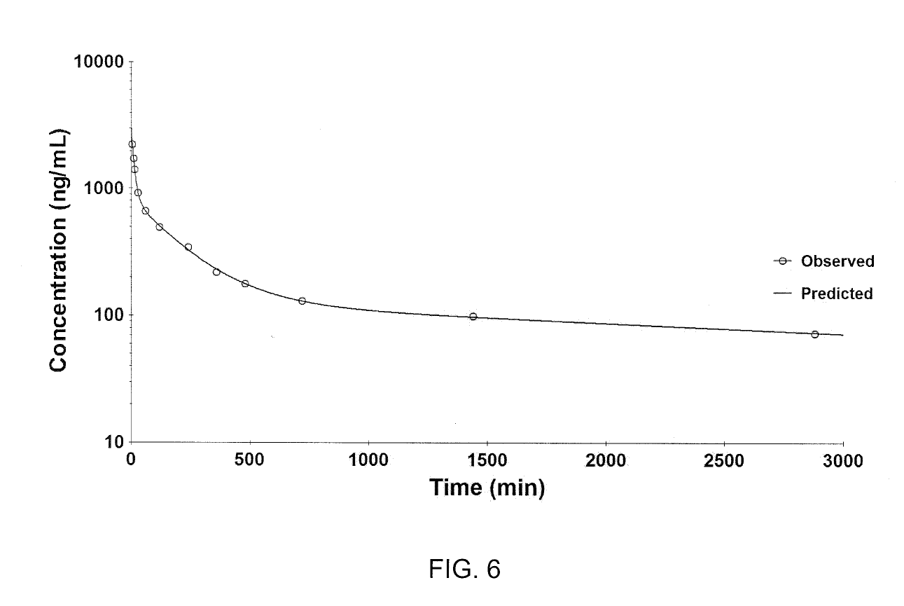

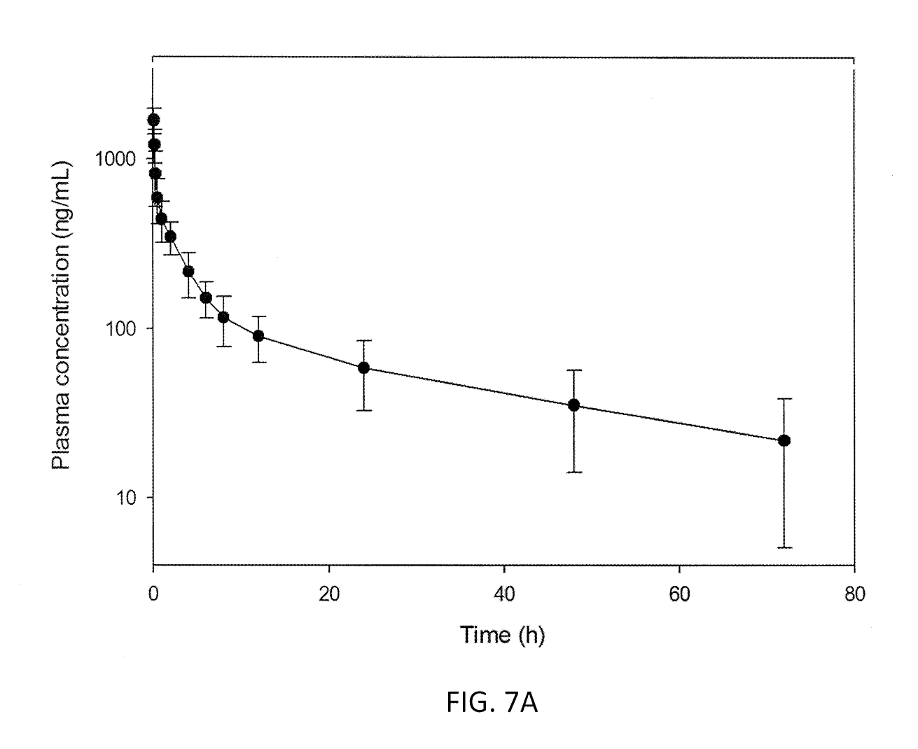

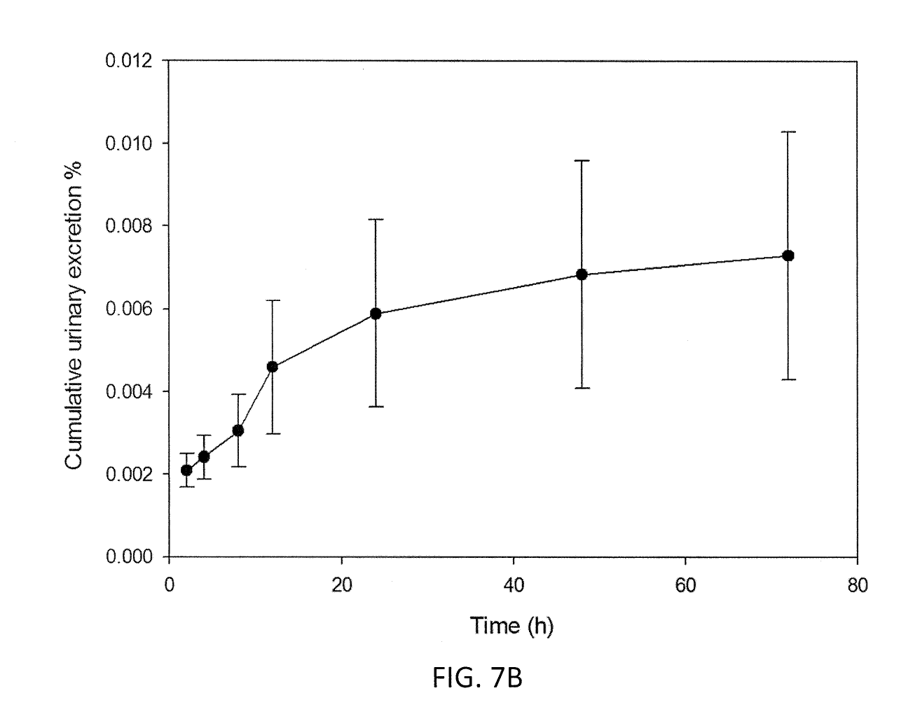

To further determine the fate of MJC13 in live animals and verify the applicability of the developed LC/MS/MS assay and formulations, pharmacokinetic studies were performed in rats. The animal experiment and protocol were reviewed and approved by the Institutional Animal Care and Use Committee at Texas Southern University. The jugular veins of eight male adult Sprague-Dawley rats (358˜377 g) were cannulated under anesthesia using a cocktail of ketamine:acetopromazine:xylazine (50:3.3:3.3 mg/kg) the day before the study. On the day of study, Formulation PT4, which includes PEG 400 and Tween 80 (1:1, v/v) with a MJC13 concentration of 7.5 mg/mL, was diluted to 1 mg/mL by normal saline. Blood and urine samples were collected from each rat right before dosing. Each rat was given a 2 mg/kg intravenous (IV) bolus dose of MJC13. Blood sample collecting tubes were heparinized and dried. In order to avoid any possible contamination, after dosing for each rat, the foremost part of each tubing associated with the needle for dosing was cut off, and then 0.5 mL of normal saline containing 20 U/mL heparin was injected to flush the tubing. Blood samples (250 μL; n=8) were collected at 5, 10, 15, 30 minutes, 1, 2, 4, 6, 8, 12, 24, 48 and 72 hours post injection, respectively. The blood samples were centrifuged at 13,000 rpm for 10 minutes and the supernatants (plasma) were obtained and immediately stored at −80° C. until analysis. Urine samples (n=4) were collected periodically, including 0-2, 2-4, 4-8, 8-12, 12-24, 24-48 and 48-72 hours post injection. After measurement of the overall urinary volume of each interval, urine samples were immediately stored at −80° C. until analysis. Within 48 hours, those plasma and urine samples were analyzed by the LC/MS/MS method of Example 1 to obtain MJC13 concentrations. After analysis, the plasma concentrations and cumulative urinary excretions of MJC13 were presented as the mean value with standard deviation (SD) and plotted against time by SigmaPlot 12.3 (Systat Software, San Jose, Calif.). (FIGS. 5, 6, 7A, and 7B).

The intravenous drug plasma concentration-time data were pharmacokinetically analyzed separately for each rat by compartmental modeling using the microcomputer-based nonlinear regression program, WINNONLIN software 5.2.1 (Pharsight Corporation, Mountain View, Calif.). A three-compartment model was provided which best described the fit for the IV bolus administration based on the appearance of the observed and predicted concentrations, the reduction in the sums of squares, and the Akaike's information criterion (Yamaoka et al., J. Pharmacokinet Biopharm., 6(2): 165-75 (1978)). The three-compartment model is described by Eq. 5:

Cₜ=A·e^{-αt}+B·e^{-βt}+C·e^{-γt}, Eq. 5

where A, B and C are the coefficients, α is the α-phase hybrid rate constant, β is β-phase hybrid rate constant, γ is the γ-phase hybrid rate constant, and Cₜ is the plasma concentration of MJC13 at time t. The pharmacokinetic parameters thus derived were the systemic or plasma clearance (Cl), the volume of distribution (Vd), the total area under the plasma drug concentration-time curve (AUC), and half-lives (T_{1/2}). The resulting main pharmacokinetic parameters are shown in Table 6. After a 2 mg/kg IV bolus injection, the MJC13 concentration reached a maximum (Cₘₐₓ) of 2708±251 ng/mL and then declined rapidly. The elimination half-life of α-phase (T_{1/2α}) was observed to be 5.64±1.30 minutes, elimination half-life of β-phase was 126±19 minutes, and elimination half-life of γ-phase was 1940±727.

TABLE 6
Pharmacokinetic parameters of MJC13 in plasma
after single IV bolus administration at 2 mg/kg to rats (n = 8).

| | |
|---|---|
| Cₘₐₓ* (ng/mL) | 2708 ± 251 |
| A* (ng/mL) | 2124 ± 222 |
| T_{1/2α}* (min) | 5.64 ± 1.30 |
| B* (ng/mL) | >483 ± 126 |
| T_{1/2β}* (min) | 126 ± 19 |
| C* (ng/mL) | 101 ± 36 |
| T_{1/2γ}* (min) | 1940 ± 727 |
| AUC_{0-∞}* (min · ng/mL) | 398685 ± 205542 |
| Cl* (mL/kg/min) | 6.04 ± 2.43 |
| V₁* (mL/kg) | 744 ± 74 |
| V₂* (mL/kg) | 2038 ± 646 |
| V₃* (mL/kg) | 8068 ± 1600 |
| Vₛₛ* (mL/kg) | 10850 ± 2129 |

*Cₘₐₓ = maximum concentration; A = coefficient of α-phase; T_{1/2α} = elimination half-life of α-phase; B = coefficient of β-phase; T_{1/2β} = elimination half-life of β-phase; C = coefficient of γ-phase; T_{1/2γ} = elimination half-life of γ-phase; AUC_{0-∞} = area under curve from time zero to infinity; Cl = total body clearance; V₁ = volume of distribution of central compartment; V₂ = volume of distribution of first peripheral compartment; V₃ = volume of distribution of second peripheral compartment; Vₛₛ = volume of distribution at steady state.

### Example 4

This example demonstrates the preparation of an intravenous pharmaceutical composition comprising MJC13 and cyclodextrin.

An excess amount of MJC13 was added to a sealed glass vial (2 ml) containing saturated (50% w/v) 2-hydroxypropyl-β-cyclodextrin (HP-β-CD) aqueous solution. The vial was shaken at room temperature for 72 hours. Thereafter, the solution was centrifuged, filtered through a 0.2 μm membrane and analyzed for MJC13 by the LC/MS/MS method of Example 1. Experiments were conducted in triplicate. The solubility of MJC13 in 50% (w/v) HP-β-CD solution was 2.6±0.3 mg/mL, which is a 10,000-fold increase compared to MJC13 itself in pure water. The result showed that MJC13 can be conveniently prepared in an aqueous solution of HP-β-CD. It is believed that the resulting pharmaceutical composition has improved biocompatibility because it lacks TWEEN 80 (polysorbate 80).

### Example 5

This example demonstrates the treatment of human prostate tumors in mice using the PT4 formulation of MJC13.

Two 22Rvl human prostate cancer xenograft studies containing four mice in each treatment group were performed. Eight mice were inoculated subcutaneously with 22Rvl prostate cancer cells. After the tumors reached an average size of 150 mm³, the mice were randomized into two groups (control and MJC13 treatment groups) of four mice each, at which time the treatments were started (Day 0). The control treatment included the same components as the PT4 formulation except that it lacked MJC13. The MJC13 (PT4 formulation) dose chosen was the maximum soluble dose (2 mg/kg). Treatments (either vehicle or formulated MJC13) were delivered intratumorally twice a week for four weeks. No toxicity was observed in mice treated with this dose intravenously.

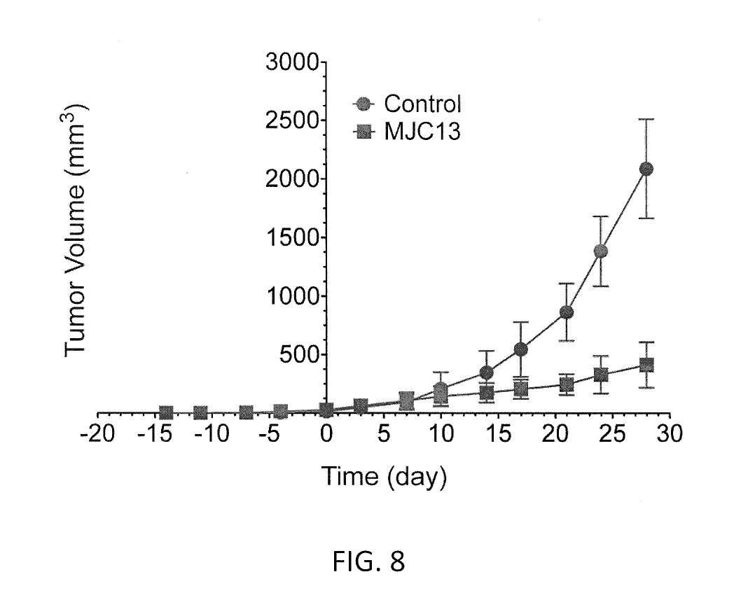

Tumor volume was measured. The results are shown in FIG. 8. As shown in FIG. 8, tumor volume decreased in mice treated with the PT4 formulation of MJC13 as compared to mice treated with control. The effect of MJC13 on tumor growth was substantial considering that the dosing regimen was only two times per week.

### Example 6

This example demonstrates a method of preparing liposomes comprising MJCI3 (non-PEGylated liposomes).

Materials:

1. MJC13 (molecular weight (MW) 272.17)

2. L-α-Phosphatidylcholine (PC) (egg PC, MW 768)

3. Cholesterol (MW 386.65)

4. Chloroform (MW 119.38)

5. Methanol (MW 32.04)

6. Normal saline

Preparation:

Into a 250 mL round bottom flask, these reactants were added in the following order:

1. 10 mg MJC13

2. 200 mg egg PC

3. 50 mg cholesterol

4. 10 mL of chloroform/methanol (9:1)

The solvent was evaporated at 60° C. in a rotating vacuum evaporator. The resulting lipid film was hydrated by 10 mL of normal saline and vortexed at 55° C. to form a suspension of multilamellar vesicles (MLVs), which was then cooled to room temperature. Small vesicles were prepared by sonication using Sonifier® S-250D digital ultrasonic processor (Branson Ultrasonics Corporation, Danbury, Conn., USA) for four minutes at amplitude=10%. To obtain small unilamellar vesicles (SUVs), the liposome suspensions were passed through the Extruder equipped with 100 nm pore-size polycarbonate filters for 10 extrusion cycles (Northern Lipids, Burnaby, BC, Canada).

### Example 7

This example demonstrates a method of preparing liposomes comprising MJC13, wherein the liposomes comprise PEG covalently attached to the phospholipid (PEGylated liposome).

The method of Example 6 was followed, except that 120 mg of 1,2-distearoyl-sn-glycero-3-phosphoethanolamine (DSPE)-mPEG5000 (MW 5800) was added into the 250 mL round bottom flask following the cholesterol and prior to the chloroform/methanol.

### Example 8

This example demonstrates the pharmacokinetic profiles of the non-PEGylated liposomes of Example 6 and the PEGylated liposomes of Example 7.

The pharmacokinetic profiles of the MJC13 conventional and PEGylated liposomes were studied in a rat model. The jugular veins of four male adult Sprague-Dawley rats (348˜383 g) were cannulated under anesthesia using a cocktail of ketamine:acetopromazine:xylazine (50:3.3:3.3 mg/kg) the day before the study.

On the day of study, three groups of rats were given three different treatments, respectively: 2 mg/kg co-solvent MJC13 formulation (PT4 of Example 2), 0.5 mg/kg non-PEGylated liposomes (prepared as described in Example 6), or 0.5 mg/kg PEGylated liposomes (prepared as described in Example 7) via IV bolus administration. Blood samples (250 μL) were collected at 5, 10, 15, 30, 60, 120, 240, 360, 480, 720, and 1440 minutes post injection, respectively. The blood samples were centrifuged at 13,000 rpm for 0.5 min. and the supernatants (plasma) were obtained and immediately stored at −80° C. until analysis. Within 48 hours, those plasma samples were analyzed by the LC/MS/MS method of Example 1 to obtain MJC13 concentrations.

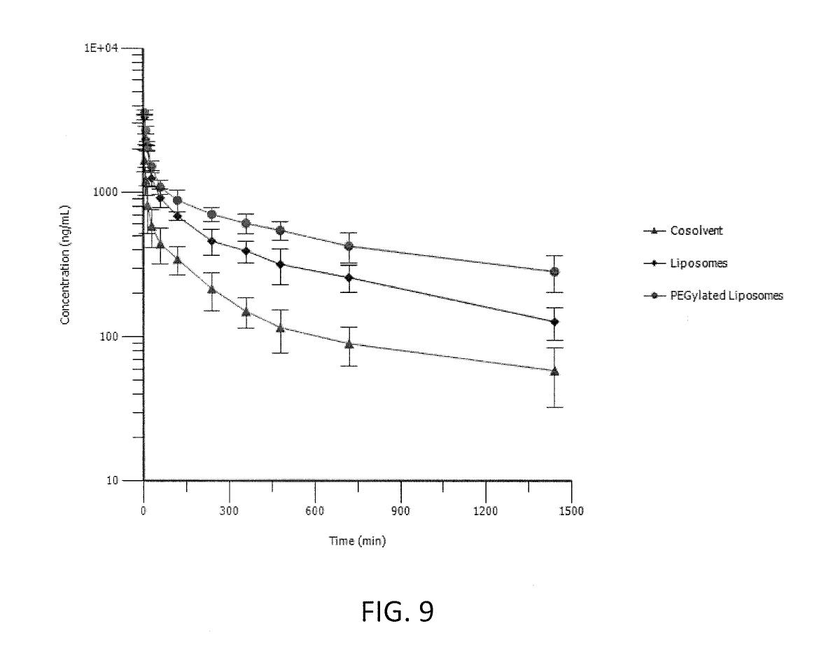

After analysis, the plasma drug concentration data were plotted against time (FIG. 9). The pharmacokinetic parameters were determined by Phoenix 1.3 (Certara, St. Louis, Mo.) with a two-compartment model which best described the fit for the IV bolus administration, based on the appearance of the observed and predicted concentrations, the reduction in the sums of squares, and the Akaike's information criterion. The final results were statistically evaluated by SYSTAT 12 (Systat Software, Chicago, Ill.) with analysis of variance (ANOVA) method (Table 7).

TABLE 7
PK parameters after single IV bolus dose of MJC13 co-solvent, conventional, and PEGylated liposomes.

| | | Co-solvent | | non-PEGylated | | PEGylated Liposomes | |
|---|---|---|---|---|---|---|---|
| | | (n = 8) | | Liposomes (n = 4) | | (n = 4) | |
| Parameters | Unit | Mean | SD | Mean | SD | Mean | SD |
| Alpha_HL | min | 8.35 | 3.12 | 11.52 | 2.31 | 11.18 | 1.19 |
| AUC | min*ng/mL | 244987 | 74886 | 576210*** | 114688 | 1043044** | 241726 |
| Beta_HL | min | 383.73 | 70.86 | 475.92 | 60.35 | 717.21** | 119.24 |
| CL | mL/min/Kg | 8.82 | 2.52 | 0.89*** | 0.18 | 0.50** | 0.12 |
| Cmax | ng/mL | 2367.60 | 224.02 | 4045.63*** | 278.62 | 4347.82* | 204.23 |
| MRT | min | 501.48 | 90.86 | 622.98 | 90.39 | 980.67** | 170.48 |
| V2 | mL/Kg | 3392.34 | 614.95 | 421.08*** | 28.81 | 360.62* | 38.09 |
| Vss | mL/Kg | 4244.21 | 651.74 | 545.09 | 32.73 | 475.80* | 41.71 |

*Statistical significance between co-solvent and PEGylated liposomes;
**Statistical significance between co-solvent and PEGylated liposomes, meanwhile statistical significance between conventional and PEGylated liposomes;
***Statistical significance between co-solvent and conventional liposomes.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A liposome comprising:
(a) a lipid bilayer;
(b) at least one liposome stabilizer; and
(c) one or more compounds selected from the group consisting of 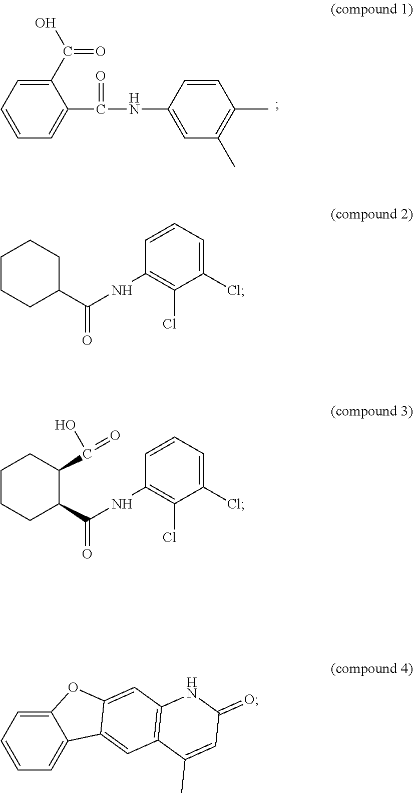 [SMILES: Cc1ccc(NC(=O)c2ccccc2C(=O)O)cc1C ; O=C(Nc1cccc(Cl)c1Cl)C1CCCCC1 ; O=C(Nc1cccc(Cl)c1Cl)[C@H]1CCCC[C@H]1C(=O)O ; Cc1cc(=O)[nH]c2cc3oc4ccccc4c3cc12]
and pharmaceutically acceptable salts, solvates, stereoisomers, derivatives, and hydrates thereof.

2. The liposome of claim 1, wherein the lipid bilayer has first and second hydrophilic surfaces and a hydrophobic interior positioned between the hydrophilic first and second surfaces, and the compound selected from the group consisting of compounds 1-4 and pharmaceutically acceptable salts, solvates, stereoisomers, derivatives, and hydrates thereof is positioned in the hydrophobic interior of the lipid bilayer.

3. The liposome of claim 1, wherein the lipid bilayer comprises phosphatidyl choline, phosphatidyl glycerol, phosphaphatidyl inositol, phosphatidyl ethanolamine, or combinations thereof.

4. The liposome of claim 1, wherein the lipid bilayer comprises one or more phospholipids selected from the group consisting of egg phosphatidylcholine (PC), egg phosphatidylglycerol (PG), soy PC, hydrogenated soy PC, didecanoylphosphatidyl choline (DDPC), dilauroylphosphatidyl choline (DLPC), dimyristoylphosphatidyl choline (DMPC), dipalmitoylphosphatidylcholine (DPPC), disaturated phosphatidylcholine (DSPC), dioctanoylphosphatidyl choline (DOPC), palmitoyl oleoyl phosphatidyl choline (POPC), decylphosphatidylcholine (DEPC), dioleoylphosphatidyl ethanolamine (DOPE), dilauroylphosphatidyl ethanolamine (DLPE), dihexanoylphosphatidyl choline (DHPC), dibutyrylphosphatidyl choline (DBPC), and combinations thereof.

5. The liposome of claim 1, wherein the at least one liposome stabilizer is selected from the group consisting of cholesterol, dimyristoylphosphatidyl glycerol (DMPG), dipalmitoyl phosphatidylglycerol (DPPG), distearoyl phosphatidylglycerol (DSPG), palmitoyl-oleoyl-phosphatidylglycerol (POPG), and combinations thereof.

6. The liposome of claim 1, comprising compound 2, egg PC, and cholesterol.

7. The liposome of claim 1, wherein the lipid bilayer comprises a phospholipid that comprises at least one poly(ethylene) glycol (PEG).

8. The liposome of claim 7, wherein the at least one PEG is selected from the group consisting of PEG 350, PEG 550, PEG 750, PEG 1000, PEG 2000, PEG 3000, PEG 5000, mPEG 350, mPEG 550, mPEG 750, mPEG 1000, mPEG 2000, mPEG 3000, mPEG 5000, and combinations thereof.

9. The liposome of claim 7, wherein the lipid bilayer comprises 1,2-distearoyl-sn-glycero-3-phosphoethanolamine-N-[methoxy(polyethylene glycol)-5000] (DSPE-mPEG 5000).

10. The liposome of claim 1, wherein the compound is compound 2.

11. A pharmaceutical composition comprising the liposome according to claim 1 and a pharmaceutically acceptable carrier.

12. A pharmaceutical composition comprising:
(a) a solvent selected from the group consisting of polyethylene glycol (PEG), alcohol, a polar aprotic solvent, and combinations thereof;
(b) a surfactant; and
(c) one or more compounds selected from the group consisting of 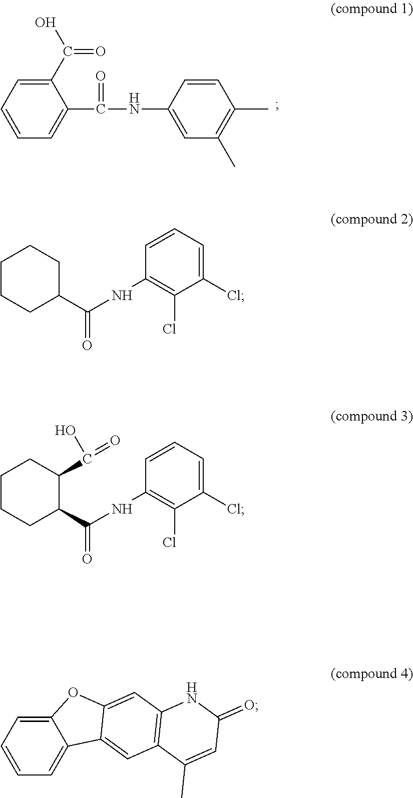 [SMILES: Cc1ccc(NC(=O)c2ccccc2C(=O)O)cc1C ; O=C(Nc1cccc(Cl)c1Cl)C1CCCCC1 ; O=C(Nc1cccc(Cl)c1Cl)[C@H]1CCCC[C@H]1C(=O)O ; Cc1cc(=O)[nH]c2cc3oc4ccccc4c3cc12]
and pharmaceutically acceptable salts, solvates, stereoisomers, derivatives, and hydrates thereof, wherein the pharmaceutical composition comprises a volume/volume (v/v) ratio of (a):(b) of from about 0.5:1 to about 1:5.

13. The pharmaceutical composition of claim 12, wherein the surfactant has a hydrophilic-lipophilic balance (HLB) of from about 10 to about 20.

14. The pharmaceutical composition of claim 12, wherein the surfactant has an HLB of from about 14 to about 17.

15. The pharmaceutical composition of claim 12, wherein the surfactant is polysorbate 20, polysorbate 40, polysorbate 60, polysorbate 80, or combinations thereof.

16. The pharmaceutical composition of claim 12, wherein the surfactant is polysorbate 80.

17. The pharmaceutical composition of claim 12, comprising the compound in a concentration of about 1 mg/mL to about 15 mg/mL.

18. The pharmaceutical composition of claim 12, comprising the compound in a concentration of about 6 to about 8 mg/mL.

19. The pharmaceutical composition of claim 12, comprising the compound in a concentration of about 7.5 mg/mL.

20. A pharmaceutical composition comprising:
(a) a solvent selected from the group consisting of polyethylene glycol (PEG), alcohol, a polar aprotic solvent, and combinations thereof;
(b) a cyclodextrin and/or a derivative thereof; and
(c) one or more compounds selected from the group consisting of 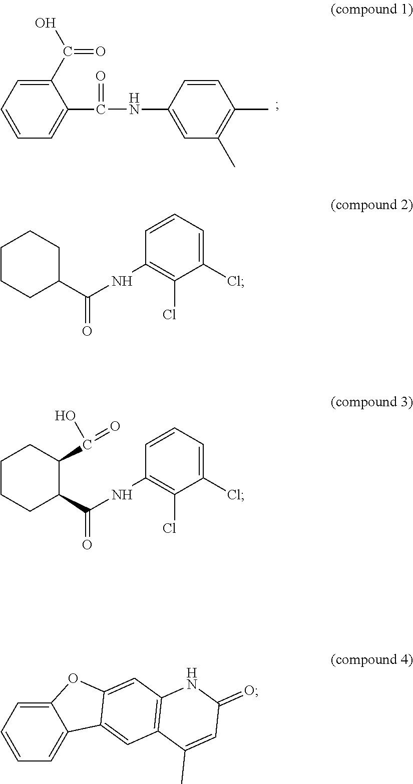 [SMILES: Cc1ccc(NC(=O)c2ccccc2C(=O)O)cc1C ; O=C(Nc1cccc(Cl)c1Cl)C1CCCCC1 ; O=C(Nc1cccc(Cl)c1Cl)[C@H]1CCCC[C@H]1C(=O)O ; Cc1cc(=O)[nH]c2cc3oc4ccccc4c3cc12]
and pharmaceutically acceptable salts, solvates, stereoisomers, derivatives, and hydrates thereof, wherein the pharmaceutical composition comprises a volume/volume (v/v) ratio of (a):(b) of from about 0.5:1 to about 1:5.

21. The pharmaceutical composition of claim 20, comprising the compound in a concentration of about 0.5 to about 10 mg/mL.

22. The pharmaceutical composition of claim 20, wherein the cyclodextrin and/or derivative thereof comprises any one or more of an α-cyclodextrin, a β-cyclodextrin, a γ-cyclodextrin, and derivatives thereof.

23. The pharmaceutical composition of claim 20, wherein the cyclodextrin and/or derivative thereof comprises hydroxypropyl-β-cyclodextrin (HP-β-CD).

24. The pharmaceutical composition of claim 20, wherein the pharmaceutical composition does not comprise a surfactant.

25. The pharmaceutical composition of claim 20, wherein the pharmaceutical composition does not comprise polysorbate 80.

26. The pharmaceutical composition of claim 12, wherein the solvent is polyethylene glycol.

27. The pharmaceutical composition of claim 12, wherein the solvent is polyethylene glycol (PEG) 200, PEG 300, PEG 400, PEG 500, PEG 600, or combinations thereof.

28. The pharmaceutical composition of claim 12, wherein the solvent is PEG 400.

29. The pharmaceutical composition of claim 12, comprising a volume/volume (v/v) ratio of (a):(b) of from about 1:1 to about 1:5.

30. The pharmaceutical composition of claim 12 comprising a volume/volume (v/v) ratio of (a):(b) of from about 1:1.

31. The pharmaceutical composition of claim 12, wherein the compound is

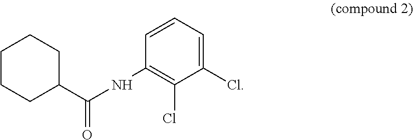 [SMILES: O=C(Nc1cccc(Cl)c1Cl)C1CCCCC1]

32. A method of inhibiting prostate or breast cancer cell growth, diminishing spermatogenesis or fertilized oocyte implantation, or treating or preventing an androgen receptor polyglutamine tract disease, prostate cancer, breast cancer, benign prostatic hyperplasia (BPH), prostatic intraepithelial neoplasia (PIN), prostatitis, enlarged prostate, or insulin-independent diabetes in a mammal, the method comprising administering the pharmaceutical composition of claim 11 to the mammal in an amount effective to inhibit prostate or breast cancer cell growth, diminish spermatogenesis or fertilized oocyte implantation, or treat or prevent an androgen receptor polyglutamine tract disease, prostate cancer, breast cancer, BPH, PIN, prostatitis, enlarged prostate, or insulin-independent diabetes in the mammal.

33. A method of detecting one or more compounds in a sample by liquid chromatography/tandem mass spectrometry (LC/MS/MS), the method comprising:
(a) preparing a sample comprising the one or more compounds;
(b) separating a first portion of the sample comprising the one or more compounds from a second portion of the sample by liquid chromatography;
(c) ionizing the first portion of the sample, separating ions according to a mass-to-charge ratio, detecting the ions, and generating one or more spectra by tandem mass spectrometry; and
(d) determining the presence of the one or more compounds in the sample when the spectra includes a mass peak associated with the compound,
wherein the one or more compounds are selected from the group consisting of 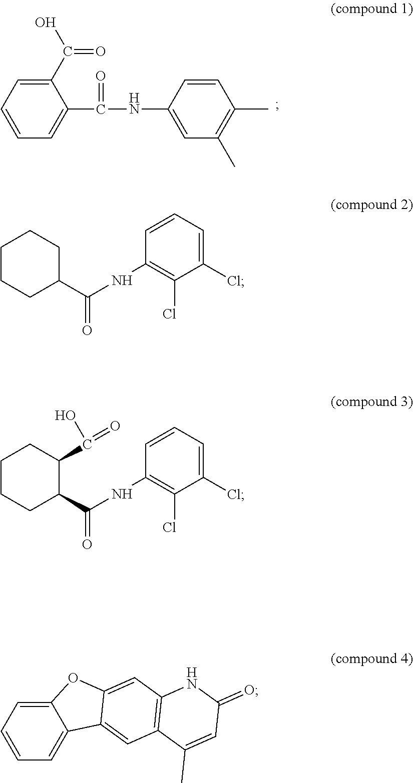 [SMILES: Cc1ccc(NC(=O)c2ccccc2C(=O)O)cc1C ; O=C(Nc1cccc(Cl)c1Cl)C1CCCCC1 ; O=C(Nc1cccc(Cl)c1Cl)[C@H]1CCCC[C@H]1C(=O)O ; Cc1cc(=O)[nH]c2cc3oc4ccccc4c3cc12]
and pharmaceutically acceptable salts, solvates, and stereoisomers thereof.

34. The method of claim 33, wherein the sample comprises blood plasma.

35. The method of claim 33, wherein the sample comprises an aqueous solution.

36. The method of claim 33, further comprising quantifying the one or more compounds in the sample.

37. The method of claim 33, comprising detecting the one or more compounds present in the sample at a concentration of about 1 picogram to about 1 nanogram.

38. The method of claim 33, wherein the compound is 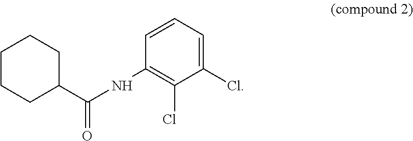 [SMILES: O=C(Nc1cccc(Cl)c1Cl)C1CCCCC1]
